(19) United States
(12) Patent Application Publication
(10) Pub. No.: US 2004/0116781 A1
(43) Pub. Date: Jun. 17, 2004

(54) Behavior based life support generating new behavior patterns from historical behavior indicators

(75) Inventor: James Mark Weaver, Austin, TX (US)

Correspondence Address: Biggers & Ohanian, PLLC, 5 Scarlet Ridge, Austin, TX, 78737, US

(73) Assignee: INTERNATIONAL BUSINESS MACHINES CORPORATION, Armonk, NY

(21) Appl. No.: 10/321,544

(22) Filed: Dec. 17, 2002

(51) Int. Cl. A61B 5/00

(52) U.S. Cl. 600/300

(57) ABSTRACT

Providing life support services to a user, including receiving a plurality of disparate historical behavior indicators, filtering the historical behavior indicators for a user in dependence upon user filter attributes, producing filtered historical behavior indicators, and identifying a behavior pattern for the user in dependence upon the filtered historical behavior indicators.

### BACKGROUND OF THE INVENTION

1. Field of the Invention

The field of the invention is data processing, or, more specifically, methods, systems, and products for behavior based life support.

2. Description of Related Art

Many conventional services are offered to people based upon broad generalizations and averages of many people's behavior over long periods of time. Conventional services do not typically offer services based upon the behavior of a single person.

A person's behavior patterns can be inferred from behavior indicating data that results from the use of many things such as credit cards, cell phones, RFID tags, bar codes and many other data generating behavior. However, much of this behavior indicating data is discarded and is not used to identify behavior patterns for a person. It would be advantageous if there were a method of to identify behavior patterns for a person and to provide life support services in dependence upon the identified behavior patterns.

### SUMMARY OF THE INVENTION

Exemplary embodiments of the invention include methods of providing life support services to a user. Exemplary embodiments include receiving a plurality of disparate historical behavior indicators, and filtering the historical behavior indicators for a user in dependence upon user filter attributes, producing filtered historical behavior indicators. Such embodiments include identifying a behavior pattern for the user in dependence upon the filtered historical behavior indicators.

In exemplary embodiments of the invention, identifying a behavior pattern for the user includes finding a set of filtered historical behavior indicators that identify a behavior pattern for the user. Such embodiments include saving the set of filtered historical behavior indicators as past behavior records for the user. In typical embodiments, finding a set of filtered historical behavior indicators that identify a behavior pattern for the user includes data mining.

Exemplary embodiments of the invention include editing the past behavior records. In such embodiments, editing the past behavior records includes receiving editing instructions from the user. Typical embodiments include establishing an action associated with the behavior pattern for the user.

The foregoing and other objects, features and advantages of the invention will be apparent from the following more particular descriptions of exemplary embodiments of the invention as illustrated in the accompanying drawings wherein like reference numbers generally represent like parts of exemplary embodiments of the invention.

### BRIEF DESCRIPTION OF THE DRAWINGS

FIG. 1 is a block diagram of architecture useful in implementing methods of providing life support services to a user.

FIG. 2 is a block diagram of data structures useful in implementing methods of providing life support services to a user.

FIG. 3 is a data flow diagram illustrating a method of providing life support services to a user.

FIG. 3a is a data flow diagram depicting a method of identifying an action to be taken in dependence upon a behavior pattern.

FIG. 4 is a data flow diagram illustrating methods of executing behavior based life support services actions.

FIG. 5 is a data flow diagram illustrating methods of executing behavior based life support services actions.

FIG. 6 is a data flow diagram illustrating a method of providing life support services to a user including identifying a heuristic.

FIG. 7 is a data flow diagram illustrating methods of identifying a discrepancy.

FIG. 8 is a data flow diagram illustrating methods of executing heuristic actions.

FIG. 9 is a data flow diagram illustrating a method of providing life support services to a user including identifying a set of default behavior indicators identifying a default behavior pattern.

FIG. 10 is a data flow diagram illustrating methods of identifying a default action in dependence upon a default behavior pattern.

FIG. 11 is a data flow diagram illustrating a method of providing life support services to a user including inferring an abstract behavior pattern.

FIG. 12 is a data flow diagram illustrating methods of inferring an abstract behavior a pattern.

FIG. 13 is a data flow diagram illustrating methods of identifying an action in dependence upon an abstract behavior pattern and executing the identified action.

FIG. 14 is a data flow diagram illustrating a method providing life support services to a user including creating a new behavior pattern.

FIG. 15 is a data flow diagram illustrating methods of creating a new behavior pattern.

FIG. 16 is a data flow diagram illustrating methods of establishing an action associated with the new behavior pattern.

FIG. 17 is a data flow diagram illustrating a method of providing life support services to a user including identifying behavior patterns from filtered historical behavior indicators.

FIG. 18 is a data flow diagram illustrating methods of identifying behavior patterns from filtered historical behavior indicators.

### DETAILED DESCRIPTION OF EXEMPLARY EMBODIMENTS

### Introduction

The present invention is described to a large extent in this specification in terms of methods for behavior based life support. Persons skilled in the art, however, will recognize that any computer system that includes suitable programming means for operating in accordance with the disclosed methods also falls well within the scope of the present invention.

Suitable programming means include any means for directing a computer system to execute the steps of the method of the invention, including for example, systems comprised of processing units and arithmetic-logic circuits coupled to computer memory, which systems have the capability of storing in computer memory, which computer memory includes electronic circuits configured to store data and program instructions, programmed steps of the method of the invention for execution by a processing unit. The invention also may be embodied in a computer program product, such as a diskette or other recording medium, for use with any suitable data processing system.

Embodiments of a computer program product may be implemented by use of any recording medium for machine-readable information, including magnetic media, optical media, or other suitable media. Persons skilled in the art will immediately recognize that any computer system having suitable programming means will be capable of executing the steps of the method of the invention as embodied in a program product. Persons skilled in the art will recognize immediately that, although most of the exemplary embodiments described in this specification are oriented to software installed and executing on computer hardware, nevertheless, alternative embodiments implemented as firmware or as hardware are well within the scope of the present invention.

### Definitions

“API” is an abbreviation for “application programming interface.” An API is a set of routines, protocols, and tools for building software applications.

“Browser” means a web browser, a communications application for locating and displaying web pages. Browsers typically comprise both a markup language interpreter, web page display routines, and an HTTP communications client. Typical browsers today can display text, graphics, audio and video. Browsers are operative in web-enabled devices, including wireless web-enabled devices. Browsers in wireless web-enabled devices often are downsized browsers called “microbrowsers.” Microbrowsers in wireless web-enabled devices often support markup languages other than HTML, including for example, WML, the Wireless Markup Language.

“Coupled for data communications” means any form of data communications, wireless, 802.11b, Bluetooth, infrared, radio, internet protocols, HTTP protocols, email protocols, networked, direct connections, dedicated phone lines, dial-ups, serial connections with RS-232 (EIA232) or Universal Serial Buses, hard-wired parallel port connections, network connections according to the Power Line Protocol, and other forms of connection for data communications as will occur to those of skill in the art. Couplings for data communications include networked couplings for data communications. Examples of networks useful with various embodiments of the invention include cable networks, intranets, extranets, internets, local area networks, wide area networks, and other network arrangements as will occur to those of skill in the art. The use of any networked coupling among television channels, cable channels, video providers, telecommunications sources, and the like, is well within the scope of the present invention.

“ESN” is an abbreviation for “Electronic Serial Number.” An ESN is a serial number programmed into a mobile communication device, such as, for example, a mobile telephone, to uniquely identify the device.

The term “field” is used to refer to data elements, that is, to individual elements of digital data. Aggregates of fields are referred to as “records” or “data structures.” Aggregates of records are referred to as “tables.” Aggregates of tables are referred to as “databases.” Records and fields in a table are sometimes referred to respectively as “rows” and “columns.” Complex data structures that include member methods as well as member data elements are referred to as “classes.” Instances of classes are referred to as “objects” or “class objects.”

A “foreign key” is a field in a first table that identifies and references a field in a second table. When such a foreign key is present the two tables are said to be “related.”

“ID” abbreviates “identification,” meaning ‘identification code’ or identification field. It is a style of reference in this disclosure to refer to user identification codes as “user IDs.” By convention in this disclosure, the field name “UserID” is used to store a user ID. Similarly, the field name “behaviorpatternID” is used to store a behavior pattern ID.

“Parlay” refers to the Open Service Access (“OSA”) Application Programming Interface (“API”) of the multi-vendor industry consortium known as the “Parlay Group.” The OSA API enables an application to access an underlying network's functionality through an open standardized interface. To allow the application to access underlying functionality, Parlay implements specific “service interfaces” and “framework interfaces.” Service interfaces access the capabilities of the underlying network. The underlying services of the network made available by the service interface are located and managed by the application through the framework interface.

“Provisioning” means providing information and instructions to carry out an LSS action. For example, provisioning call blocking onto an SCP means providing the information and instructions to an SCP to implement call blocking on a telephone number.

“PSTN” is an abbreviation for “Public Switched Telephone Network.” PSTN refers to an international telephone system based on copper wires carrying analog voice data. Telephone service carried by the PSTN is sometimes called “plain old telephone service.”

The OSGi stands for “Open Services Gateway Initiative.” OSGi is a programming framework based on Sun Microsystems Java programming for deployment of applications to OSGi compatible devices, such as small memory devices. Applications for OSGi compatible devices are packaged as “bundles” and are installed on an OSGi service framework. The bundles are downloaded to the OSGi compatible devices, swapped in and out of the service framework by the OSGi compatible devices, and dynamically updated by the OSGi compatible devices.

“Server” in this specification refers to a computer or device comprising automated computing machinery on a network that manages resources and requests for access to resources. A “web server,” or “HTTP server,” in particular is a server that communicates with browsers by means of HTTP in order to manage and make available to networked computers documents in markup languages like HTML, digital objects, and other resources.

A “Service Control Point (SCP)” is an interface to a telecommunication provider's database containing subscriber services information and call routing information.

A “SLEE server” is a server operating portable telecommunication services and application frameworks in a JAIN SLEE compliant execution environment. “JAIN” refers to the JAVA API for Integrated Networks. SLEE servers in typical embodiments of the present invention are implemented in JAVA using the JTAPI, the Java Telephony API. “JAIN SLEE,” or the JAIN Service Logic Execution Environment, an element of Sun Microsystems' industry-oriented de facto standard JAIN initiative, is a set of interfaces and standards for telecommunications and Internet operations within carrier grade telecommunications networks and Internet networks. JAIN-compliant telecommunications services are tested and deployed in the JAIN Service Logic Execution Environment.

“Smart Card” means a small electronic device, typically, about the size of a credit card, that contains electronic memory. Smart cards often contain an embedded integrated circuit. Smart cards are used for a variety of purposes, including storing a patient's medical records, purchasing, employee ID badges, RFID tags, and any other use that will occur to those of skill in the art. A smart card reader reads information from a smart card, and writes information to a smart card.

“SMS” is an abbreviation for Short Message Service. SMS is a service for sending short text messages to mobile phones.

“SS7” means Common Channel Signaling System No. 7 (i.e., SS7 or C7) is a global standard for telecommunications defined by the International Telecommunication Union (ITU) Telecommunication Standardization Sector (ITU-T). The standard defines the procedures and protocol by which network elements in the public switched telephone network (PSTN) exchange information over a digital signaling network to effect wireless (cellular) and wire line call setup, routing and control.

“TCP/IP” means the Transmission Control Protocol (TCP) and the Internet Protocol (IP) operating together. TCP/IP is a packet switching protocol. TCP establishes a virtual connection between a data source and a data destination. IP specifies that data will be sent from the source to the destination in packets and IP specifies the addressing scheme of the source and the destination. TCP monitors the delivery of the data and the order in which the packets are delivered.

“Telephony” means functions for translating sound into electrical signals, transmitting them, and then converting them back to sound. The term “telephony” is used to refer to computer hardware and software that performs functions traditionally performed by telephone equipment.

“WAP” refers to the Wireless Application Protocol, a protocol for use with handheld wireless devices. Examples of wireless devices useful with WAP include mobile phones, pagers, two-way radios, and hand-held computers. WAP supports many wireless networks, and WAP is supported by many operating systems. Operating systems specifically engineered for handheld devices include PalmOS, EPOC, Windows CE, FLEXOS, OS/9, and JavaOS. WAP devices that use displays and access the Internet run “microbrowsers.” The micrbrowsers use small file sizes that can accommodate the low memory constraints of handheld devices and the low-bandwidth constraints of wireless networks.

“Websphere®” refers to the “Websphere®” application server available from International Business Machines Corporation. WebSphere is a Java technology-based application server with self-contained, modular applications called “Web Services.” The Web Services include applications for security, clustering, connectivity, and scalability.

### Behavior Patterns for Users

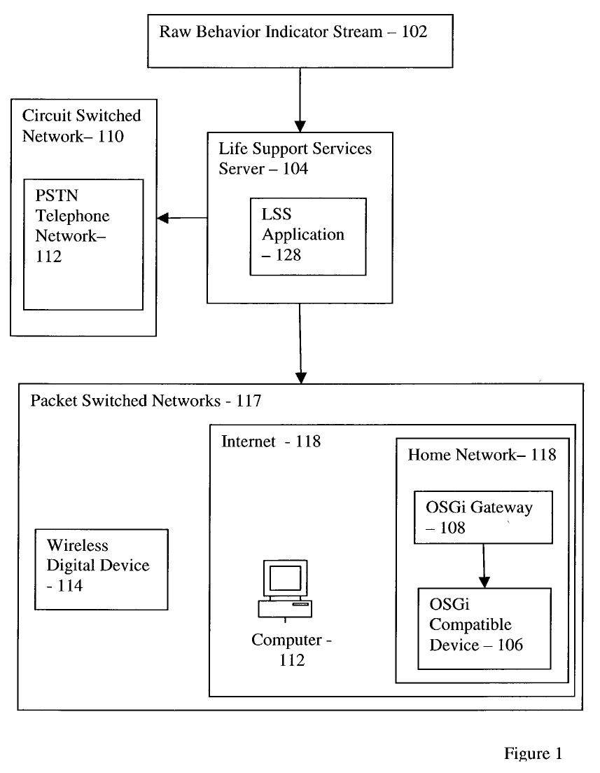

FIG. 1 is a block diagram showing an overall architecture for information handling systems useful in implementing various exemplary embodiments of the present invention. The architecture of FIG. 1 includes a Life Support Services (LSS) server (104). The Life Support Services Server (104) is automated computing machinery that manages resources and requests for access to resources on behalf of an LSS application (128) installed and operating on the LSS server (104).

The LSS application (128) of FIG. 1 is application software running on the LSS Server (104) that implements methods of providing life support services (LSS) to a user in accordance with the present invention. The LSS application (128) filters a stream (102) of raw behavior indicators into working cache memory for a particular user. The LSS application (120) identifies a behavior pattern record for the user in dependence upon the filtered behavior indicators and also in dependence upon records of past actions. The LSS application identifies and executes, in dependence upon the behavior pattern record for the user, a current, behavior-based LSS action.

The LSS application (128) receives a stream (102) of raw behavior indicators through the LSS Server (104). Behavior indicators are data provided by a service provider from which an actor's behavior can be inferred. For example, information from a single credit card purchase can include information of the location of the point of sale, what was purchased, the time the purchase was made, and the amount of the purchase, each of which is a behavior indicator of an actor, in this example, a credit card purchaser. Continuing with the credit card example, credit card purchases on December 24^{th,} at 7:30 p.m., at a shopping center may indicate that the actor is last-minute Christmas shopping. Last-minute Christmas shopping can identify a defined behavior pattern in an LSS application. An example of a behavior-based LSS action in response to identifying such a behavior pattern, could be to download a copy of the shopper's Christmas list to the shoppers PDA.

Behavior indicators are generated as a result of many kinds of behavior. Examples of behaviors that result in the generation of behavior indictors include making credit card purchases, moving people and things tracked by bar code systems, RFID systems, or GPS systems, logging onto computers, swiping personnel identification badges at work, making telephone calls, receiving telephone calls, passing toll tags under toll booths, checking in at an airport terminal for a flight, and any other behavior as will occur to those of skill in the art which results in the generation of computer data describing behavior that can be streamed to an LSS application. In this specification, such data describing behavior are referred to as “behavior indicators.” Behavior indicators include, purchase price, purchase time, purchase location, location of cars, locations of watches, locations of people, times and days people log onto computers, times and days people receive telephone calls, the telephone numbers people call, the telephone numbers from which people receive calls, and any other behavior indicator that will occur to those of skill in the art.

The behavior indicator stream of FIG. 1 is provided by various service providers. One example of a behavior indicator, as mentioned above, is the location of a mobile telephone. An example of a service provider that can stream mobile telephone locations to an LSS application (128) is AT&T Wireless. More particularly, the “Find Friends” service provided by AT&T Wireless provides to third parties, such as an LSS application (128) of the present invention, the location of a registered wireless phone to the nearest cell phone tower.

A single service provider can gather and stream many different types of behavior indicators. For example, Citibank offers a smart card called the GSA Card. The GSA Card is a smart card that acts as an employee ID badge, a web server access ID, a building access ID, a standard credit card, and a standard calling card. The GSA Card also holds digital certificates, and holds user medical information. The service provider for the Citibank GSA Card can collect and stream to an LSS application (128) behavior indicators such as web server access times, telephone numbers called with the calling card feature, behavior indicators generated as a result of credit card purchases, and behavior indicators related to medical information.

The LSS server (104) of FIG. 1 is coupled for data communication with a circuit switched network (110) and a packet switched network. While the distinction between circuit switched networks and packet switched networks may seem arbitrary, discussing the architecture of FIG. 1 in the context of circuit switched networks and packet switched networks provides an opportunity to introduce protocols and operating environments useful in implementing various embodiments of behavior based LSS services.

Circuit-switched networks (110) are networks in which data is sent from a source to a destination over a dedicated physical path between the source and destination. An example of a circuit-switched network is the PSTN network (112) providing telephone service. SS7 (Signal System 7) protocols provide the controlling infrastructure of the PSTN network. SS7 defines a standard for messages, a protocol for out-of-band signaling, and an intelligent network topology needed for advanced telephony services. SS7 defines procedures and protocol by which network elements exchange information over a digital signaling network to effect wireless (cellular) and wireline call setup, routing, and control.

By contrast to a circuit-switched network, a packet-switched networks are networks in which data is divided into packets and each packet is sent individually from the source to the destination without obtaining a dedicated connection between the source and destination. An example of a packet-switched network (117) is the internet (118). “TCP/IP” is the standard protocol for the internet. TCP/IP is actually two protocols, the Transmission Control Protocol (TCP) and the Internet Protocol (WP), operating together. TCP establishes a virtual connection between a data source and a data destination and monitors the delivery of the data and the order in which the packets are delivered. IP specifies that data will be sent from the source to the destination in packets and specifies the addressing scheme of the source and the destination.

The architecture of FIG. 1 advantageously allows the LSS application (128) to execute behavior based LSS actions over both circuit switched and packet switched networks. One way in which the LSS Server (104) provides resources to the LSS application (128) to execute behavior based LSS actions over both circuit switched and packet switched networks is through SLEE. In this case, the LSS Server operates as a SLEE Server.

A SLEE server is a server operating portable telecommunication services and application frameworks in a JAIN SLEE compliant execution environment. SLEE is oriented to event driven applications. SLEE applications receive requests for services in the form of events. A SLEE (or a SLEE server) has a logical event router that receives a SLEE event emitted by an event producer, identifies a SLEE component interested in the event, and delivers the event to the SLEE component.

One way to build an event driven application is to provide a single event handler method to receive all events. When such an event handler receives an event, it inspects the event and directs further processing of the event with a switch statement, switching on the event type of the event. The switch statement implements the event routing logic within the application.

SLEE applications comprise components known as Service Building Block or ‘SBB’ components. Each SBB component is identified with event types accepted by the component. Each SBB component has event handler methods that contain application code that processes events of these event types. An SBB component may be a root SBB component or child SBB component. A root SBB component typically represents a complete service. A child SBB component typically represents a function within the complete service of the root SBB. For example, an application developer may develop a root LSS SBB for a particular user. The application developer may create children SBB's for behavior based LSS actions to be executed in dependence upon identifying a behavior pattern for a user.

Another way the LSS Server (104) can support the LSS application's (128) execution of behavior based LSS actions over both circuit switched networks and packet switched networks (117) is through Parlay. Parlay is an API that supports applications across both circuit switched networks such as the PSTN network, and packet switched networks such as the internet. Parlay defines an architecture that enables an application to access an underlying network's (e.g., PSTN network's) functionality through an open standardized interface.

To allow the application to access underlying functionality, Parlay implements specific “service interfaces” and “framework interfaces.” Service interfaces access the capabilities of the underlying network. An example of a capability of an underlying PSTN network is call routing. An example of an underlying capability in a wireless network supporting WAP is messaging. The underlying services of the network made available by the service interface are located and managed by the application through a framework interface.

Descriptions of SLEE and Parlay in this disclosure are for explanation, not for limitation. Whether to implement any particular embodiments in an execution environment is optional, and to the extent that any execution environment is utilized, there are a number of them that will work with various embodiments of the present invention. Methods of behavior based LSS according to embodiments of the present invention may be implemented, for example, in SLEE environment, a Parlay environment, in a Websphere® environment, or in any other execution environment as will occur to those of skill in the art.

In the architecture of FIG. 1, the LSS server (104) is coupled for data communication with a PSTN telephone network (112). An example of a behavior based LSS action using the architecture of FIG. 1, is provisioning a call blocking function on a telephone number in the PSTN telephone network (112), in dependence upon identifying a behavior pattern for the user, such as the user being asleep.

In the architecture of FIG. 1, the LSS server (104) is coupled for data communication with a computer (112). An example of a behavior based LSS action using the architecture of FIG. 1 is sending an email from the LSS application (128) to the computer (112) through the internet (118) in dependence upon identifying a behavior pattern for the user.

In the architecture of FIG. 1, the LSS server (104) is coupled for data communication with a home network (118) containing an OSGi Gateway (108) and OSGi Compatible Device (106), such as a coffee pot. The LSS application (128) running on the LSS server (104) may download OSGi compatible bundles to the OSGi compatible devices through the internet (118), home network (118) and OSGi Gateway (108) to execute a behavior based LSS action, such as such as turning off the coffee pot, in dependence upon a behavior pattern of the user, such as the user leaving home and going to work.

In architecture of FIG. 1 the LSS server (104) is coupled for data communication with a wireless digital device (114), such as a mobile telephone or PDA. The LSS application (128) may send an SMS message to the wireless digital device (114) using the WAP to execute a behavior based LSS action in dependence upon identifying a user behavior pattern for the user, such as last minute Christmas shopping. WAP refers to the Wireless Application Protocol, a protocol for use with digital wireless devices (114) such as mobile phones, pagers, two-way radios, and hand-held computers. WAP supports many wireless networks, and WAP is supported by many operating systems such as PalmOS, EPOC, Windows CE, FLEXOS, OS/9, and JavaOS.

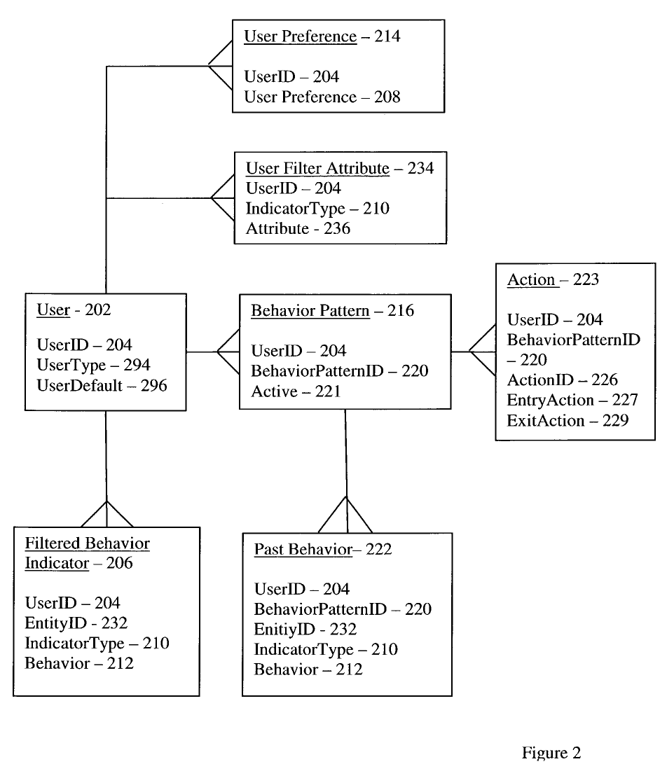

FIG. 2 is a block diagram of exemplary data structures useful in implementing typical embodiments of behavior based LSS services according to the present invention. The data structures of FIG. 2 include user records (202) having a userId field (204) to identify the user. The user record (202) represents a user or subscriber of LSS services. A behavior based LSS action is executed for the user in dependence upon identifying a behavior pattern for the user.

The data structures of FIG. 2 include filtered behavior indicator records (206). The filtered behavior indicator records (206) represent behavior indicators of the user or behavior indicators of another entity whose behavior affects the user. Because the filtered behavior indicator records (206) are particularly associated with a user, the filtered behavior indicator records (206) are related many-to-one to the user record (202) through a userlD field (204) used as a foreign key.

The filtered behavior indicator records (206) of FIG. 2 include an entity field (232). The entity field represents the entity whose behavior resulted in the generation of the filtered behavior indicator record (206). The entity producing the filtered behavior indicator record may be the user or another entity associated with the user, that is, an entity other than the user as such. For example, a user may have authority to receive behavior indicators of the user's children, such as, the location information of the child's GPS wristband or RFID wristband. The behavior indicators of the user's children are filtered for the user and LSS services are provided for the user in dependence upon the filtered behavior indicators of the user's children. The entity does not have to be a person. In fact, many entities associated with a user are things. For example, the location of a user's tracked mail is a behavior indicator associated with the user. In this specification, an entity ‘associated’ with the user is a person or thing identified by an entityID field (232) in a filtered behavior indicator record (206) related to a user record (202) for a particular user.

The filtered behavior indicator records (206) of FIG. 2 include an indicator type field (210). The indicator type represents a kind of behavior indicator represented by the filtered behavior indicator. For example, indicator types include credit card purchase price, credit card purchase location, mobile phone location, GPS location of a car, orientation information provided by RFID tags such as whether a person is lying down or sitting up, or any other type of filtered behavior indicator (206). The filtered behavior indicator records (206) include a behavior field (212) representing the value of the filtered behavior indicator record (206). For example, if the filtered behavior indicator type (210) is a GPS location of a car, then the location coordinates of the car are stored in the behavior field (212). Because the filtered behavior indicator records (206) represent behavior indicators of many different kinds, the fields of the filtered behavior indicator records (206) will vary according to type of filtered behavior indicator record (206) as will occur to those of skill in the art.

The data structures of FIG. 2 include user filter attribute records (234). The user filter attribute records (234) represent filtering criteria used to filter a raw behavior identifier stream (reference 102 in FIG. 1) for a particular user. Because the filtering attribute records (234) represent filtering criteria for a particular user, the filtering attribute records (234) are related to the user record (202) many-to-one through the userID field (204) used as a foreign key. The user filter attribute record (234) includes an indicator type (210) identifying the type of behavior indicator in the behavior indicator stream (reference 102 in FIG. 1) filtered for the user. The behavior filter attribute record includes an attribute field (236). The attribute field represents an attribute identifying the particular user associated the behavior indicator.

The data structures of FIG. 2 include a behavior pattern record (216). The behavior pattern records (216) represent patterns of behavior for the user. A pattern of behavior for the user is not limited to patterns of the user's behavior. In fact, a pattern of behavior for the user may include behavior indicators produced by other entities associated with the user. Because the behavior pattern records (216) represent particular behavior patterns for a user, the behavior pattern records (216) are related to the user records (202) many-to-one through the userID field (204) used as a foreign key. A behaviorpatternID field (220) identifies each behavior pattern record (216) for a user. The behavior pattern record (216) includes an active field (221). The active field (221) is a boolean indicator used to indicate whether the behavior pattern record (216) represents a current behavior pattern for a user.

The data structures of FIG. 2 include past behavior records (222). Each past behavior record (222) represents a particular past behavior of a user or a particular past behavior of an entity associated with the user. A set of past behavior records (222) identify a particular behavior pattern for the user. Because a set of past behavior records (222) identify a particular behavior pattern for the user, the past behavior records (222) are related to the behavior pattern records (216) many-to-one through the userID field (204) and the behaviorpatternID field (220) used as a composite foreign key.

The past behavior record (222) of FIG. 2 also includes an entityID field (232) representing the entity associated with the user whose past behavior is represented by the past behavior record (222). The past behavior record (222) of FIG. 2 also includes an indicator type (210) representing the type of behavior indicator stored in the past behavior record (222). The past behavior indicator record (222) includes a behavior field (212) indicating the value of the behavior indicator represented by the past behavior record (222).

The data structures of FIG. 2 include action records (223). The action records represent actions to be taken in dependence upon identifying a behavior pattern record (216) for the user. Because more than one action may be executed for a particular behavior pattern record (216) for the user, the action records (223) are related many-to-one to the behavior pattern records (216) through a userID field (204) and a behaviorpatternID field used as a composite foreign key. The action records (223) include an actionID field representing a behavior-based LSS action to be executed in dependence upon identifying a behavior pattern (216). The action records (223) also include an ‘EntryAction’ field (227) as a Boolean indication whether an action is an entry action for a behavior pattern. The action records (223) also include an

‘ExitAction’ field (229) as a Boolean indication whether an action is an exit action for a behavior pattern.

An entry action is an action to be executed when a user's behavior is first identified as matching a behavior pattern, that is, the user is said to ‘enter’ a behavior pattern. After a user's behavior is first identified as matching a behavior pattern, as filtered behavior indicators continue to arrive in working cache for the user, multiple additional matches for the behavior pattern often will continue to be identified. There is usually no need, however, to repeatedly perform actions for the behavior pattern merely because the pattern continues to be matched. For this reason, it is advantageous in many embodiments of the present invention to statefully distinguish whether a user has ‘entered’ or ‘exited’ a pattern.

An exit action is an action to be executed when a user's behavior is identified as no longer matching a behavior pattern, that is, the user is said to ‘exit’ a behavior pattern. Exit actions can affect, terminate or reverse, processes or conditions established by the entry actions, or they can establish or carry out processes or conditions unrelated to the entry actions. An example of an entry action is provisioning call blocking instructions onto an SCP to block calls to a user's home telephone number in dependence upon identifying a behavior pattern that the user is asleep. An example of an exit action is provisioning instructions onto an SCP to remove the call blocking from a user's home telephone number when the behavior pattern of the user being asleep no longer represents a current behavior pattern for the user, because the user is awake and preparing to go to work.

The data structures of FIG. 2 include user preference records (214). The user preference records represent preferences (214) of the user. For example, a user preference may be that the user likes blues music. The user's preference for blues music is stored in the user preference field (208). Because a user may have multiple preferences, the user preference records (214) are related to the user record (202) many-to-one through a userID field (204) used as a foreign key.

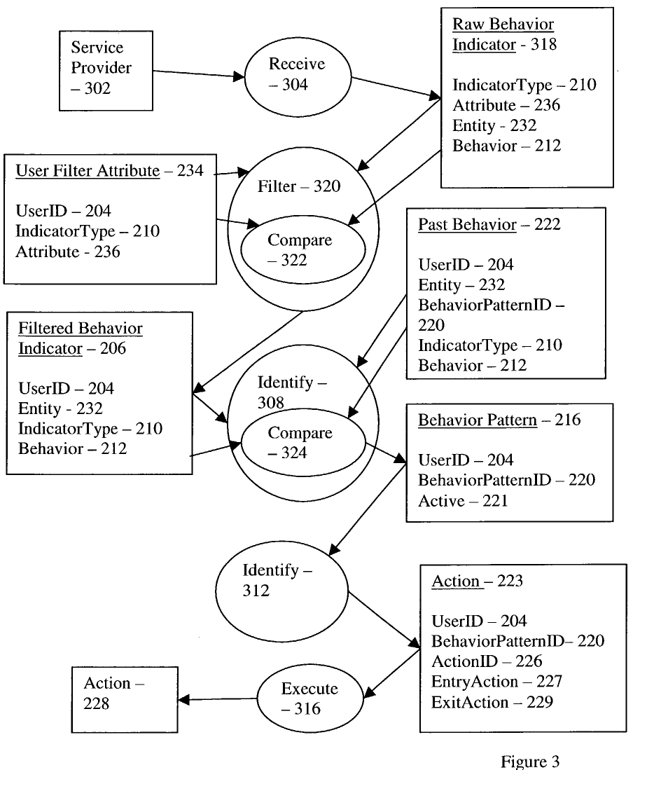

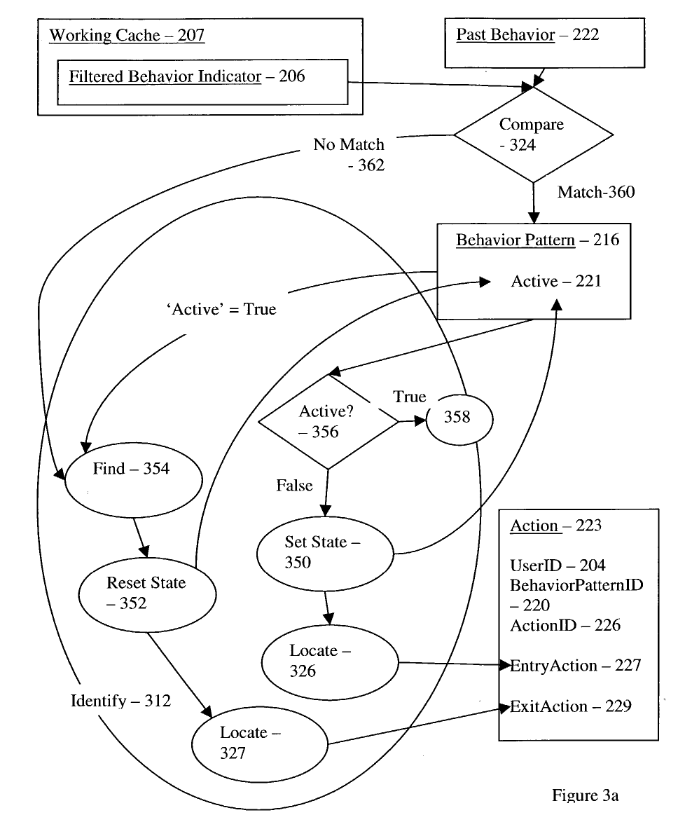

FIG. 3 illustrates a method of providing life support services to a user. The method of FIG. 3 includes receiving (304) a raw behavior indicator stream having a plurality of disparate raw behavior indicators. The disparate raw behavior indicators are represented in FIG. 3 by raw behavior indicator records (318) received (304) from various service providers (302). The term ‘disparate’ behavior indicators (318) means that the raw behavior indicator records (318) represent behavior indicators of different types, such as for example, behavior indicators (318) representing credit card purchases, GPS locations, computer log on times, or any other type of behavior indicator that will occur to those of skill in the art. Because each raw behavior indicator record (318) may represent a different type of behavior indicator, the behavior indicator record (318) of FIG. 3 includes an indicator type field (210) to identify the type of behavior indicator represented by the behavior indicator record (318).

The raw behavior indicator records (318) of FIG. 3 are also behavior indicators associated with many different users. Because the raw behavior indicator records (318) are for many different users, the raw behavior indicator records (318) include an attribute field (236). The attribute field (236) represents a particular attribute by which a particular user or entity associated with the user may be identified. For example, the attribute may be a credit card number, telephone number, ESN number of a mobile phone, or any other attribute particularly linking the raw behavior indicator record (318) to a user or entity associated with the user.

The method of FIG. 3 includes filtering (320) the behavior indicators (318) in dependence upon user filter attribute records (234). The user filter attribute records (234) represent filtering criteria for a user. Because the user filter attribute records (234) represent filtering criteria for a user, the user filter attribute records (234) include a userID field (204) identifying the user. The user filter attribute records (234) include an indicator type field (210) and an attribute field (236) representing the criteria used to filter the raw behavior indicator records (318). That is, the raw behavior indicator records (318) are filtered by at least indicator type (210) (indicating the type of behavior indicator), and attribute (236) (information linking the behavior indicator to the user).

In many life support services systems according to embodiments of the present invention, a filter process includes data conversion. Because the raw behavior indicator records (318) are of various types and originate with various service providers, the raw behavior indicator records are received in various data formats. Comparing behavior indicators with user filter attributes and comparing filtered behavior indicators with past actions are both advantageously accomplished with predetermined internal data structures, rather than trying to program for all the various structures of the raw indicators. In embodiments of the present invention, therefore, filtering (320) the behavior indicators (318) typically includes converting the data structure of the raw behavior indicator records into a predetermined data structure used internally within embodiments of the present invention. Because different types of raw behavior indicators include different information, the predetermined internal data structures are organized to be appropriate for each type of converted raw behavior indicator. For example, a raw behavior indicator record received for a credit card transaction is typically converted into an internal credit card transaction data structure including fields for customer ID, credit card number, expiration date, purchase price, and transaction time.

In the method of FIG. 3, filtering (320) the raw behavior indicator records (318) for a user includes comparing (322) the fields of the raw behavior indicator records (318) converted to a consistent data structure with the fields of the user filter attribute records (234). If the indicator type field (210) and the attribute field (236) of the behavior indicator record (318) match the indicator type field (210) and attribute field (236) of the user filter attribute record (234), then the raw behavior indicator qualifies as a filtered behavior indicator for a user. The userID field (204) in the user filter attribute records (234) identifies the user.

Filtering (320) the raw behavior indicators (318) for a user may be carried out by an LSS application (reference 128 of FIG. 1) running on an LSS server (reference 104 of FIG. 1) operating as a SLEE server. One way of filtering (320) that exploits the use of the SLEE environment for carrying out the steps of providing behavior based LSS services includes performing the step of filtering the raw behavior indicators (318) outside of the SLEE environment. By filtering outside of the SLEE environment, the portion of the LSS application (reference 128 of FIG. 1) carrying out the step of filtering (320) the raw behavior indicator records (318) is used as a SLEE event producer. The step of filtering the raw behavior indicators (318) results in a filtered behavior indicator record (206) which is used as a SLEE event.

The filtered behavior indicator record (206), used as a SLEE event, is delivered to a root LSS SBB component by a SLEE logical event router. A root LSS SBB component for each user is instantiated in the SLEE environment and receives the filtered behavior indicator record (206) and carries out the remaining steps of providing LSS for a user.

The method of FIG. 3 includes identifying (308) a behavior pattern record (216) for a user in dependence upon the filtered behavior indicators records (206) and past behavior records (222). By identifying a set of past behavior records (222) that match the filtered behavior indicator records (206) in cache memory, a behavior pattern record (216) for the user is identified. The behavior pattern is identified because the set of past behavior records (222) are related to the behavior pattern record many-to-one to through a userID field (204) used as a foreign key.

In typical embodiments of the present invention, identifying a behavior pattern for a user further comprises comparing filtered behavior indicators with past behavior records. As shown for the method according to FIG. 3, identifying (308) a behavior pattern record (216) for a user includes comparing (324) the fields of filtered behavior indictor records (206) maintained in working cache with the fields of a set of past behavior indicator records (222). If the comparison of the fields of filtered behavior indicator records (206) and the fields of past behavior records (206) reveal a match in the entity fields (206), the indicator type fields (210), and the behavior fields (212) then the set of past behavior indicator records identifies a behavior pattern. A related behavior pattern record (216) is identified by locating the behavior pattern record (216) related to the set of past behavior records (222) one-to-many through a userild and behaviorpatternID field used as a foreign key. For each filtered behavior indicator (206) filtered into working cache, another comparison is made between the filtered behavior indicator records (206) and the past behavior records (222). If the filtered behavior indicator records (206) match a set of past behavior records (222), the behavior pattern record (216) related to the set of past behavior records (222) is identified.

Comparing (324) the fields of the filtered behavior indictor records (206) in working cache memory with the fields of a set the past behavior indicator records (222) does not have to result in a perfect match to identify (308) a behavior pattern (216) for a user. Most behavior patterns are not that finely defined. Therefore, the degree to which the filtered behavior indicator records (206) must match the past behavior indicator records (222) to identify a behavior pattern record (216) depends of various factors such as the tolerances of the methods used for comparing (324) the type of information contained in the filtered behavior indicator records (206) and the past behavior records (222), the accuracy and precision used in generating the filtered behavior indicator records (206) and the past behavior indicator records (222), user specified conditions, and other factors that will occur to those skilled in the art.

The behavior patterns according to embodiments of the present invention advantageously in many embodiments are stateful. ‘Stateful’ means that applications according to of embodiments of the present invention set at least one attribute of a pattern in computer memory so that the attribute remains available from time to time or from event to event. More particularly, in embodiments of the present invention, when ‘events’ correspond to arrival a behavior indicator in a current working buffer, it is of interest generally to know when a pattern match results, whether the pattern so matched was also matched on an immediately previous event, therefore indicating that the user or actor of the pattern is still ‘in’ the pattern. To the extent that a user has previously ‘entered’ a pattern, that is, a pattern record representing a behavior pattern was already identified for the user by comparing filtered behavior indicators in working cache with past behaviors, the entry actions for the pattern have already been performed, so that, so long as the pattern continues to be matched on event after event, there is no need to repeat entry actions every time the same pattern is matched.

The method of FIG. 3 includes identifying (312) an action to be taken in dependence upon a behavior pattern. Actions to be taken in dependence upon a behavior pattern are represented in FIG. 3 by action records (223). Because actions are taken in dependence upon a behavior pattern, the action records (223) of FIG. 3 are related many-to-one to the behavior pattern record (216) through the userID field (204) and the behaviorpatternID field (220) used as a composite foreign key.

FIG. 3a sets forth a data flow diagram more particularly depicting a method of identifying (312) an action to be taken in dependence upon a behavior pattern. Behavior pattern records (216) either represent current behavior patterns for a user or they do not, and whether they do can be represented in data elements describing state. When comparison (324) indicates that filtered behavior indicator records (206) in working cache memory (207) match (360) a set of past behavior indicator records (222) related to a behavior pattern record (216), that behavior pattern record (216) represents a current behavior pattern for a user. In the example of FIG. 3a, if the ‘active’ field in the pattern record (216) so identified is set ‘false,’ the method includes setting (350) the ‘active’ field (221) to ‘true’ and then locating (326) for execution action records (223) identified, by a Boolean field such as ‘EntryAction (227) set to ‘true,’ as entry actions. On subsequent arrival of filtered behavior records (206) resulting in matches (360) for the same behavior pattern (216), ‘active’ (221) is already set to ‘true,’ so no action is taken (358).

In the example of FIG. 3a, the process of identifying (312) is activated also when filtered behavior indicators arrive and no match (362) is found for a pattern record based upon the filtered behavior indicators currently in a working cache (207). In this circumstance, identifying (312) actions includes finding (354) all the pattern records for the user of the working cache, having ‘active’ (221) set to ‘true.’ Such records represent previously active behavior patterns, no longer active, whose exit actions have not yet been executed. The method of FIG. 3a includes finding such records, resetting their ‘active’ fields (221) to ‘false,’ and locating (327) for execution action records (223) identified, by a Boolean field such as ‘ExitAction (229) set to ‘true,’ as exit action.

For clarity of explanation, stateful administration of behavior patterns and actions is described also in terms of dynamic state changes: A field named ‘active’ (221) in the behavior pattern record (216) is a boolean indicator used to indicate whether the behavior pattern record (216) represents a current behavior pattern for a user. When a behavior pattern record (216) is first found to represent a current behavior pattern for a user, the active field (221) is set (350) to ‘true.’ For each filtered behavior indicator (206) filtered into a working cache, another comparison (324) is made between the filtered behavior indicator records (206) and the past behavior records (222) to identify any behavior pattern records (216) that represent current behavior patterns for a user. When the behavior pattern record (216) no longer represent a current behavior pattern for a user, the active field (221) is reset (352) to ‘false.’

Typical embodiments of the present invention include executing an identified action, that is, an action identified in dependence upon a behavior pattern. The method of FIG. 3, for example, includes executing (316) the identified behavior based LSS action (228). One way of implementing the step of executing (316) an identified action (223), is deploying a child SBB for the identified action in a SLEE environment. The child LSS SBB component is a component directed to a specific action, such as, sending an email. A root LSS SBB component for the user carries out the step of identifying (312) the action by locating an actionID (226) in an action record (223) related to an identified behavior pattern record (216). For example, a root LSS SBB component for the user may carry out the step of identifying a behavior pattern for the user indicating that the user is at work, logged onto the user's work computer, and the user's mail has arrived. The root LSS SBB carries out the step of locating actionID, such as EmailMailNoticeWork, identifying a behavior based LSS action, such as, sending an email to the user telling the user that the mail has arrived. The root LSS SBB fires a SLEE event to the child SBB component called EmailMailNoticeWork causing the child SBB component called EmailMailNoticeWork to carry out the action of emailing the user a notice that the mail has arrived.

Another way of executing (316) the identified action is to use a service interface in Parlay. For example, if a set of filtered behavior indicators indicate that the user is in a behavior pattern of sleeping. A behavior based LSS action may be to initiate call blocking on the user home telephone. A service interface in Parlay carries out the step of provisioning call blocking instructions onto a service control point in a telephone network to have calls to the user's telephone number blocked.

An alternate way of executing (316) the identified behavior based LSS action (228) is through an action object created by calling a factory method in an action factory class. Such a method of executing (316) an action to be executed is illustrated by the following pseudocode:

Action a=ActionFactory.createActionObject (actionID);

a.takeAction(userID);

The member method ActionFactory.createActionObject(actionID) is a factory method defined in the following pseudocode for an exemplary action factory class:

//
// Action Factory Class
//
// Defines a parameterized factory method for creating action objects
//
class ActionFactory
{

public static Action createActionObject(actionID)
{

Action anAction = null; // establish pointer or reference for new object
switch(actionID)
{

| case 1: anAction = new Action1; break; | |
|---|---|
| case 2: anAction = new Action2; break; | |
| ... ... ... | |
| case N-1: | anAction = new ActionN-1; break; |
| case N: | anAction = new ActionN; break; |

} // end switch( )
return anAction;

} // end createActionObject( )

} // end class ActionFactory

The exemplary member method ActionFactory.createActionObject(actionID) is a parameterized factory method that functions by creating a new concrete action class object selected in dependence upon the action ID provided as a parameter. Set forth below are examples of action classes that are useful, for example, in carrying out actions or commands selected by users in response to identifying LSS actions (228) to be executed. The first example is an abstract action used to define a common interface for concrete actions classes.

//
// abstract action class
//
class abstract Action
{

//
// declare virtual function, define in subclasses
//
public abstract boolean takeAction( ) == 0;

}

The exemplary concrete action class set forth just below is a pseudocode example of a concrete action class having a member method that carries out the exemplary LSS action of notifying a user by email at work that the user's mail has arrived.

// concrete action class for action ‘EmailMailNoticeWork’
//
class Action EmailMailNoticeWork: Action
{

public boolean takeAction(userID)
{

boolean success = false;
success = EmailMailNoticeWork(userID,
“Your mail is in your box in the mail room.”);
return success;

}

}

In this example, a life support application according to the present invention matched behavior indicators for a user with past behaviors identifying a behavior pattern in which the user is at work, logged on, and the user's mail has arrived in the mail room. The member method takeActiono in this example attempts to email to the user the message that the user's mail is now in the user's box in the mail room, returning to the calling application a Boolean indication 'success' whether the email message was sent successfully.

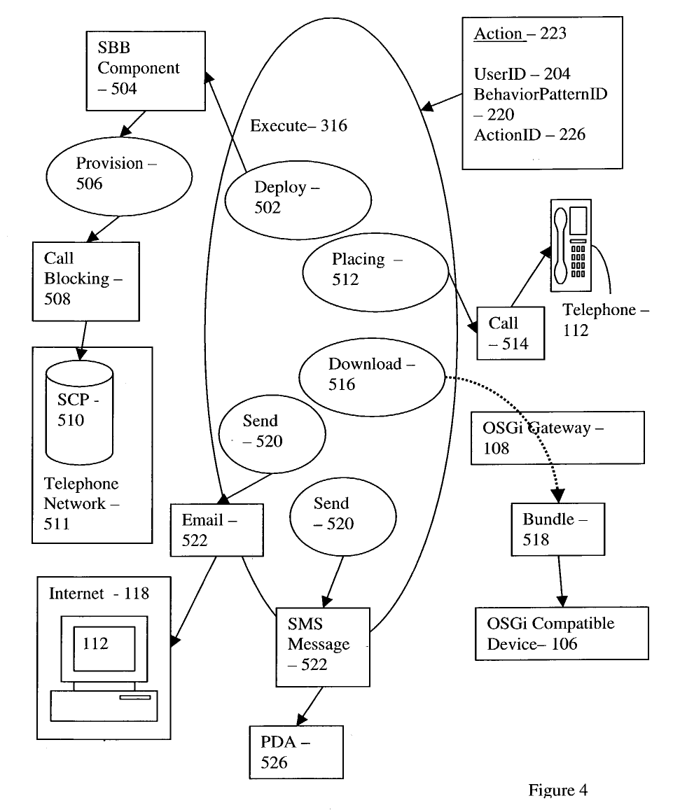

FIG. 4 illustrates a method of executing (316) an identified action (223). The method of FIG. 4 includes deploying (502) an SBB component (504) in a SLEE environment. For example, an LSS action for call blocking may be executed in dependence upon the user being asleep. A root LSS SBB component for the user deploys a child SBB CallBlocking component for call blocking in dependence upon identifying a behavior pattern for the user indicating the user is asleep. The SBB CallBlocking component (504) provisions (506) call blocking instructions (508) onto a service control point (SCP) (510) in a telephone network (511) to block calls to the users telephone number.

The method of FIG. 4 includes downloading (516) OSGI compliant bundles (518) through an OSGI gateway (108) to an OSGi compatible device. Examples of OSGi compatible devices are home entertainment systems, home appliances, and home outlets. An example executing (316) a behavior based LSS action in dependence upon identifying a behavior pattern for a user is downloading OSGi compliant bundles to a coffee pot to turn off the coffee pot in dependence upon identifying a behavior pattern of a user traveling to work.

The method of FIG. 4 includes placing (512) a telephone call (514). In some instances, in dependence upon identifying a behavior pattern for a user that indicates an emergency, executing (316) an LSS action includes placing (512) a call (514) to emergency services. One way of carrying out placing (512) a telephone call (514) is by using a service interface in Parlay. A specific service interface places the call.

The method of FIG. 4 includes sending (520) an email (522) and sending (524) an SMS message (526). For example, in dependence upon a behavior pattern indicating that the user is at work and that the tracked mail has arrived, executing (316) a behavior based LSS action many include sending (522) an email to the user at work notifying the user that the mail has arrived or sending (522) an SMS message on the user's PDA notifying the user that the mail has arrived.

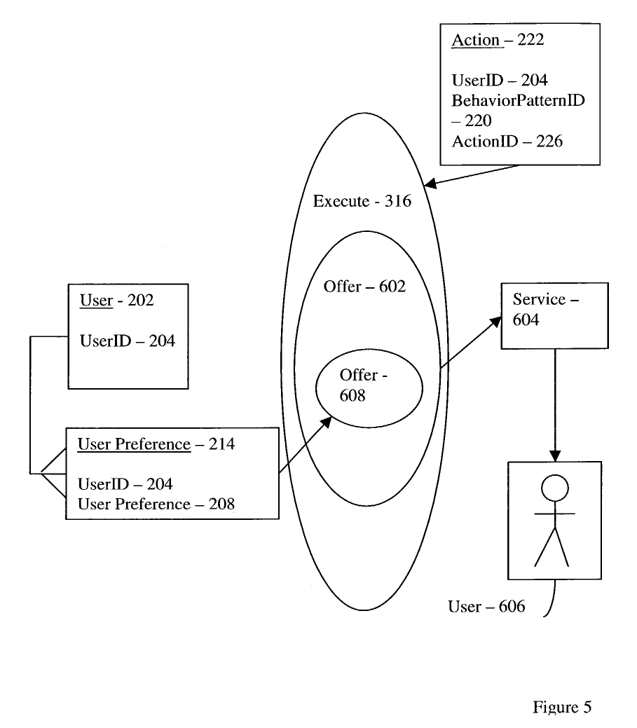

FIG. 5 illustrates a method of executing (316) an identified action (228). The method of FIG. 5 includes offering (602) a service (604) to a user (606). In some cases, a user may not want the execution of a behavior based LSS action to result in the execution of a service on the user's behalf. Instead, the user may want the, behavior based LSS action to offer the user a service. For example, a user who is stuck in traffic may not want the LSS action of calling the user's house to notify his family. Instead, the user may want the action executed to offer (602) a service (604) to the user (606). The user may then decide on a case-by-case basis whether to call home. Offering (602) a service (604) to the user may include sending an offer by email, sending an offer by SMS message, sending an offer by fax, calling the user, or any other method of offering a service that will occur to those of skill in the art.

In the method of FIG. 5, offering (602) a service (604) includes offering (608) a service (604) in dependence upon user preferences (208) in a user preference record (214). User preferences (208) represent the user's interests and thus, the user preference record is related to the user record many-to-one through the userID field (204) used as a foreign key. By offering (608) a service (604) in dependence upon user preferences (208) services may tailored specifically for the user. For example, if the behavior pattern for the user indicates that the user is on a usual trip to Chicago, and the user preference records indicate that the user is a fan of blues music, and enjoys hot dogs, then an LSS service may be to offer to make the user reservations at a Chicago blues club and provide directions to Chicago hot dog stands.

### Identifying Heuristics from Discrepancies

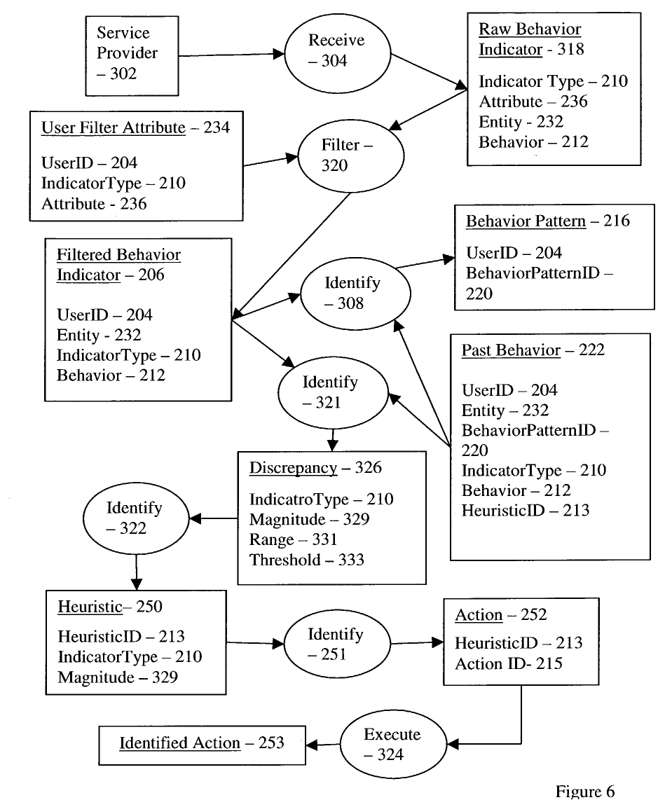

FIG. 6 is a data flow diagram illustrating an exemplary method of providing life support services to a user. The method of FIG. 6 includes receiving (304) a plurality of disparate behavior indicators. As discussed above with reference to FIG. 3, the disparate behavior indicators are represented in data structures as records such as the raw behavior indicator records (318) of FIG. 6. The term ‘raw’ means that the raw behavior indicators are not filtered for a user and that they are in a variety of data formats depending upon their types and upon the service providers where they originate. The term ‘disparate’ behavior indicators (318) means that the raw behavior indicator records (318) represent behavior indicators of different types, such as for example, behavior indicators (318) representing credit card purchases, GPS locations, computer log on times, or any other type of behavior indicator that will occur to those of skill in the art. The raw behavior indicator records (318) of FIGS. 6 are also behavior indicators associated with many users.

The method of FIG. 6 includes filtering (320) the behavior indicators (318) for a user in dependence upon user filter attributes. As discussed above with reference to FIG. 3, filtering (320) the behavior indicators for a user in dependence upon user filter attributes typically includes converting raw behavior indicators into a predetermined internal data structure and comparing the converted raw behavior indicator record (318) with user filter attribute records (234).

The method of FIG. 6 includes identifying (308) a behavior pattern (216) for a user in dependence upon the filtered behavior indicators (206) and a plurality of past behavior records (222). As discussed above with reference to FIG. 3, in typical embodiments of the present invention, identifying (308) a behavior pattern includes comparing filtered behavior indicator records (206) with past behavior records (222). When the filtered behavior indicator records (206) match the set of past behavior records, then identifying a behavior pattern includes locating the behavior pattern record (216) that is related to the set of past behavior records (222). The identified behavior pattern record (216) is said to represent a current behavior pattern for a user.

Identifying (308) a behavior pattern for a user does not require a perfect match between the filtered behavior indictor records (206) and the set the past behavior indicator records (222). The degree to which the filtered behavior indicator records (206) must match the past behavior indicator records (222) to identify a behavior pattern for the user depends of various factors such as the tolerances of the methods used for comparing (324) the filtered behavior indicator records (206) and the past behavior records (222), the type of information contained in the filtered behavior indicator records (206) and the past behavior records (222), the accuracy and precision used in generating the filtered behavior indicator records (206) and the past behavior indicator records (222), user specified conditions, and other factors that will occur to those skilled in the art.

The method of FIG. 6 includes identifying (321) a discrepancy (326) between the filtered behavior indicators (206) and the plurality of past behavior records (222). A discrepancy between the filtered behavior indicators (206) and the past behavior records (222) indicates that in fact a filtered behavior indicator (206) is not perfectly matched with one of the set of past behavior records (206) identifying a current behavior pattern for the user.

In this specification, discrepancies between the filtered behavior indicators (206) and the past behavior records (222) are represented in data structures by records such as, for example, the discrepancy records (326) of FIG. 6. The discrepancy records (326) of FIG. 6 include an IndicatorType field (210) representing the type of discrepancy record (326). The discrepancy record type is the same as the type code in, for example, an IndicatorType field (210) for the filtered behavior indicator record (206) and past behavior record (222) demonstrating a discrepancy. The discrepancy records (326) include a ‘Magnitude’ field (329) representing the magnitude of the discrepancy (326) between the filtered behavior indicators (206) and the past behavior records (222).

The method of FIG. 6 includes identifying (322) a heuristic (250) in dependence upon the discrepancy (326) between the filtered behavior indicators (206) and the plurality of past behavior records (222). A “heuristic” is a common-sense rule drawn from experience to solve problems. In this specification, heuristics are represented in data structures by records such as the one illustrated at reference (250) in FIG. 6. For practical implementation, heuristics are given effect through actual actions. Actions to be carried out to give effect to heuristics are represented in data by records such as reference (252) in FIG. 6. In this example, more than one heuristic may be identified for each discrepancy, therefore, particular actions to be carried out to vindicate a particular heuristic are identified by a heuristic identification code such as for example heuristic ID (213), used as a foreign key in a one to many relationship with the heuristic records (250). The heuristic record includes an indicator type field (210) identifying the type of behavior indicator having a discrepancy and a magnitude field (329) identifying the magnitude of discrepancy.

In many cases, heuristics are helpful to the user. The following example is provided for clarity of explanation. A plurality of filtered behavior indicators in working cache for a user include behavior indicators identifying that the user is logged on at work, the user's ID badge at work was scanned at 8:00 a.m., GPS locations of the user's car is in the employee parking lot at of the user's office building. The filtered behavior indictors and the past behaviors match sufficiently to identify a behavior pattern for the user that the user is at work. However, there is a discrepancy between the filtered behavior indicators and the past behaviors. The filtered behavior indicators for the user's mobile phone indicate that the user's mobile phone is at the user's house. An example of a heuristic identified in dependence upon a discrepancy is a heuristic identifying the discrepancy as the location of the mobile phone. An example of an action giving effect to the heuristic is contacting the user and notifying the user that the mobile phone is at home.

The method of FIG. 6 includes identifying (251) an action (252) to be taken in dependence upon the heuristic (250). Action records (252) represent the actions that give effect to an identified heuristic (250). The action records (252) of FIG. 6 include a heuristic ID (213) identifying the identified heuristic that is given effect, through the action represented in the action record. The action records (252) also include an actionID field (215) identifying an action to give effect to the heuristic. More than one action may give effect to a single heuristic, and therefore, the action records (252) are related to the heuristic records (250) many-to-one through the heuristicid field (213) used as a foreign key.

The method of FIG. 6 includes executing (324) the identified action (253). Executing (324) the identified action (253) gives effect to the identified heuristic. Executing (342) the identified action (253) includes executing an action identified by the actionid field (215) of the action record. Executing (342) the identified action may include deploying an SBB component in a SLEE environment, deploying an interface in a Parlay environment, creating an object by calling a factory method in a heuristic action class, or any other method of executing an identified action that will occur to those of skill in the art.

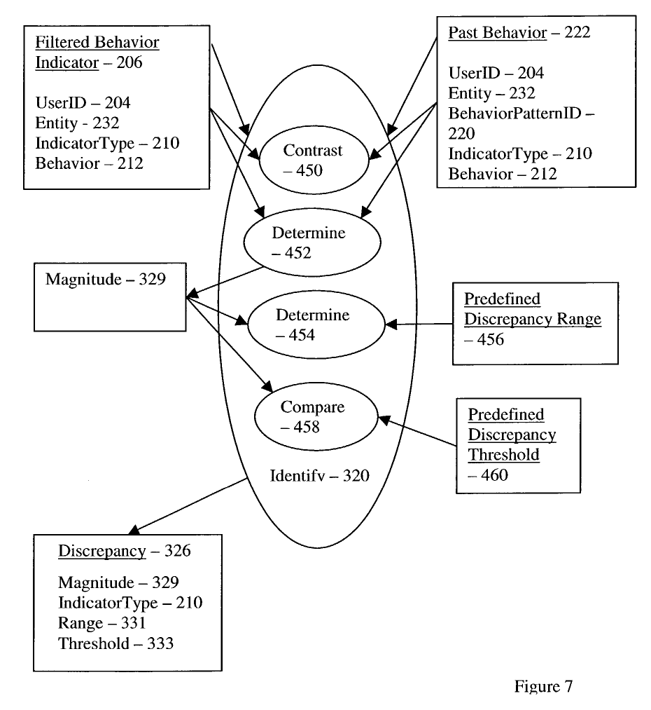

FIG. 7 is a data flow diagram illustrating a method of identifying (320) a discrepancy (326) between the filtered behavior indicators (206) and the plurality of past behavior records (222). The method of FIG. 7 includes contrasting (450) attributes of past behavior records (222) and attributes of filtered behavior indicators (206). In this specification, the attributes of past behaviors and attributes of filtered behaviors are represented as fields in data structures such as the fields in the filtered behavior indicator records (206) and the fields in the past behavior records (222) of FIG. 7.

In typical embodiments of the present invention, contrasting (450) the filtered behavior indicators and past behavior indicators includes identifying filtered behavior indicator records (206) and past behavior records (222) having the same value of IndicatorType (210) and the same value of entity field (232), thereby advantageously contrasting indicators and past behaviors of the same type for the same entity. Contrasting filtered behavior indicators and past behaviors of the same type and from the same entity is useful in identifying a heuristic for the user because the discrepancy is an indication of the degree to which the filtered behaviors (206) are not a perfect match with the past behavior records (222). Contrasting (450) the filtered behavior indicators and past behavior indicators also typically includes finding a difference between the behavior fields (212) of the filtered behavior indicator records and the past behavior records.

In the method of FIG. 7, identifying (320) a discrepancy (326) includes determining (452) a magnitude (329) of the discrepancy (326). The magnitude (329) of the discrepancy is used to indicate the size of the difference between the filtered behavior indicator and the past behavior. One way of determining (452) a magnitude (329) includes subtracting the behavior field (212) of the filtered behavior record (206) from the behavior field (212) of the past behavior indicator. Because the values in the behavior field (212) of the filtered behavior indicator record (206) may be larger than the value of the past behavior record (222) an absolute value of the result of the subtraction is used as the magnitude. Continuing with the pseudo code from above, the following lines of pseudo code are an example of determining (452) a magnitude (329) of the discrepancy.

Contrasting (450) filtered behavior indicators and past behaviors and determining (452) a magnitude (329) of a discrepancy (326), if there is one, can be carried out as illustrated in the following segment of pseudocode. ‘Pseudocode’ refers to examples of source code presented for purposes of explanation only, not for limitation. There is no representation that pseudocode can be compiled or executed. Many ways of programming methods according to the present invention will occur to those of skill in the art, in addition to the way illustrated by pseudocode, and all such ways are well within the scope of the present invention.

while(FilteredBehaviorIndicator=getNext(CurrentWorkingCache))!=EOF)

{

PastBehavior = getNext(PastBehaviorRecord,
FilteredBehaviorIndicator.IndicatorType,
FilteredBehaviorIndicator.Entity);
if ((Magnitude = PastBehaviorValue.Behavior -

FilteredBehaviorIndicator.Behavior) != 0)

{

Discrepancy aDiscrepancy = new Discrepancy( );
Discrepancy.setMagnitude(abs(Magnitude));
Discrepancy.setIndicatorType(
FilteredBehaviorIndicator.IndicatorType);

}

}

The pseudocode example scans the working cache record by record, reading in turn each filtered behavior indicator in the cache. For each filtered behavior indicator in the cache, the call

PastBehavior=getNext(PastBehaviorRecord,

FilteredBehaviorIndicator.IndicatorType,

FilteredBehaviorIndicator.Entity);

finds a past behavior record of matching type and entity. The pseudocode example then determines whether a discrepancy exists by contrasting the behavior values of the filtered behavior and the past behavior. More particularly, the pseudocode example carries out contrasting by subtracting the behavior values in the following parameter of the ‘if’ statement:

Magnitude=PastBehaviorValue.Behavior−FilteredBehaviorIndicator.Behavior.

The pseudocode treats a discrepancy as existing if the Magnitude is non-zero. If the pseudocode so finds a discrepancy, it creates a new discrepancy record by the call to

Discrepancy aDiscrepancy=new Discrepancy( ), treating the discrepancy record as a new class object of type ‘Discrepancy’ and setting the values of Magnitude and IndicatorType in the new discrepancy record by calls to setMagnitude( ) and setlndicatorType( ).

In some filtered behavior records (206) and past behavior records (222), the values in the behavior field (212) may not be in a form for subtraction to provide a useful magnitude. When behavior values are not amenable to subtraction, determining (452) a magnitude (329) includes using functions specific to the IndicatorType (210) field of the filtered behavior indicator records (206) and the past behavior indicator records (222) to find a magnitude. For example, if the filtered behavior indicator records (206) and the past behavior records (222) represent locations, and coordinates are stored in the behavior field, one way of determining a magnitude is using Pythagoras' theorem to calculate the distance between the coordinates in the filtered behavior indicator record and the coordinates in the past behavior record. In other cases, the behavior fields (212) of the filtered behavior indicators and the past behavior records are converted to a form to facilitate subtraction. For example, time values may be converted from local time to universal time, or from civilian to military time designations, to facilitate subtraction.

In the method of FIG. 7, identifying (320) a discrepancy (326) between the filtered behavior indicators (206) and the plurality of past behavior records (222) includes determining (452) a magnitude (329) of the discrepancy (326), and determining (454) whether the magnitude (329) is within a predefined range (456) for the discrepancy (326). Methods of determining (454) whether the magnitude (329) is within a predefined range include subtracting the magnitude from the upper limit of the range. If the difference is positive, then the magnitude (329) is below the upper limit of the range. Methods of determining (454) whether the magnitude (329) is within a predefined range also include subtracting the magnitude from the lower limit of the range. If subtracting the magnitude from the lower limit yields a negative value, then the magnitude is within the predefined range. If the magnitude is both below the upper limit of the predefined range and above the lower limit of the predefined range, then the magnitude is within the predefined range.

Predefined ranges provide guidelines to identify appropriate heuristics. For example, if the discrepancies indicate that the user is following a common path home from work because the filtered behavior indicator records match the past behavior records for physical locations, but there are discrepancies in the filtered behavior records and the past behavior records for time values, then a predetermined range is helpful in identifying a heuristic. If the magnitude of the discrepancy in time is within a first range, an identified heuristic may be to do nothing. If the magnitude is within another predefined range, an identified heuristic may be to contact the user and ask if the user is stuck in traffic. If the magnitude is in a still further predefined range, an identified heuristic may be to take another action such as contacting the user and downloading an alternate route home to the user on the user' PDA.

In the method of FIG. 7, identifying (320) a discrepancy (326) between the filtered behavior indicators (206) and the plurality of past behavior records (222) includes determining (452) a magnitude (329) of the discrepancy (326), and comparing (458) the magnitude (329) with a predefined threshold (460) for the discrepancy (326). Methods of comparing (458) the magnitude (329) of the discrepancy (326) with a predefined threshold (460) include subtraction. If subtracting the magnitude from the threshold results in a positive value, then the magnitude is below the threshold. If subtracting the magnitude from the threshold results in a negative value, then the magnitude is above the threshold.

A predefined threshold provides additional information for identifying a heuristic in dependence upon the discrepancy. For example, a threshold may be used to identify a drastic heuristic. Returning to the example of the user following a common path home from work, if the magnitude of the discrepancy in time passes a predetermined threshold because the user has come to a stop, a heuristic may be to call a tow truck. Comparing the magnitude with a predefined threshold may also be used in conjunction with determining if the magnitude is within a predefined range.

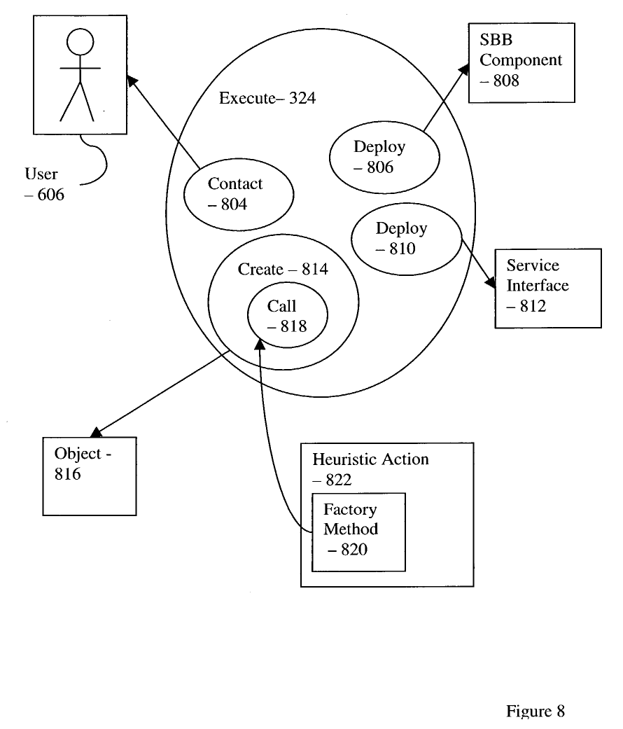

FIG. 8 is a data flow diagram illustrating methods of executing (324) an identified action (253). One method for executing an action according to FIG. 8 includes contacting (804) a user (606). Contacting (804) a user (606) can include, for example, calling the user on a telephone, sending the user an email, sending the user a fax, sending the user an SMS message, downloading a message to the user, or any other method of contacting (804) a user that will occur to those of ordinary skill in the art.

Another method of executing (324) an identified action according to FIG. 8 comprises deploying (806) an SBB (808) component in a SLEE environment. One way of deploying (806) an SBB (808) component in a SLEE environment is to deploy a child SBB component that carries out an action that gives effect to an identified heuristic. For example, a child SBB component may send the user an email notifying the user that the user's mobile telephone is at home, when a root SBB for the user identifies a behavior pattern for the user that the user is at work, and identifies discrepancies in locations of the mobile phone between the filtered behavior indicator records and past behavior records. Another way of executing (324) the identified action comprises deploying (810) a service interface (812) in a Parlay environment. Alternately, executing (324) the identified action is carried out in a Webshere® environment, or any other environment that will occur to those of ordinary skill in the art.

A still further method of executing an identified action according to FIG. 8 comprises creating (814) an object (816) by calling (818) a factory method (820) in a heuristic action class (822). Such a method of executing (324) an action is illustrated by the following pseudocode, a two-line segment for inclusion in calling application code:

HeuristicAction a=HeuristicActionFactory.createHeuristicActionObject(actionID);

a.takeHeuristicAction(userID);

As described earlier with refernence to FIG. 6, embodiments of the present invention typically identify (251) actions (252) for a heuristic (250) through a shared heuristicID (213) used as a foreign key. The two-line segment just above therefore advantageously can be set in a small loop that will carry out the two-line segment once for each action (252) for a heuristic (250).

The member method HeuristicActionFactory.createActionObject(actionID) is a factory method defined in the following pseudocode for an exemplary action factory class for an identified heuristic:

//
// Heuristic Action Factory Class
//
// Defines a parameterized factory method for creating action objects that give effect
to identified heuristics
//
class HeuristicActionFactory
{

public static HeuristicAction createHeuristicActionObject(actionID)
{

// establish pointer or reference for new object
HeuristicAction aHeuristicAction = null;
switch(actionID)
{

| case 1: alleuristicAction = new Action1; break; | |
|---|---|
| case 2: alleuristicAction = new Action2; break; | |
| ... ... ... | |
| case N-1: | aHeuristicAction = new ActionN-1; break; |
| case N: | aHeuristicAction = new ActionN; break; |

} // end switch( )
return aHeuristicAction;

} // end createHeuristicActionObject( )

} // end class HeuristicActionFactory

The exemplary member method

HeuristicActionFactory.createHeuristicActionObject(actionID)

is a parameterized factory method that functions by creating a new concrete heuristic action class object selected in dependence upon the action ID provided as a parameter. Set forth below is an abstract action used to define a common interface for concrete action classes that are useful, for example, in carrying out actions or commands selected by developers to give effect to identified heuristics.

//
// abstract heuristic action class
//
class abstract HeuristicAction
{

//
// declare virtual function, define in subclasses
//
public abstract boolean takeHeuristicAction( ) == 0;

}

Concrete heuristic action classes having member methods to give effect to identified heuristics are used to carry out an identified heuristic action giving effect to an identified heuristic. For example, a concrete heuristic action class for a heuristic action to email a user can be implemented according to the following pseudocode:

// concrete action class for action ‘EmailNoticeWorkDiscrepancyMobilePhone’
//
class HeuristicAction EmailNoticeWorkDiscrepancyMobilePhone: Action
{

public boolean takeHueristicAction(userID)
{

boolean success = false;
success = EmailNoticeWorkDiscrepancyMobilePhone (userID,
“Your mobile phone is at home.”);
return success;

}

}

In this example, a life support application has matched behavior indicators and past behaviors identifying a behavior pattern representing that a user is at work, also finding a discrepancy indicating that the user's mobile phone is still at the user's home. The member method takeHeuristicAction( ) in the pseudocode segment just above attempts to email to the user a message that the user's mobile phone is at home, returning to the calling application a Boolean indication ‘success’ whether the email message was sent successfully.

### Identifying Default Behavior Patterns for a User

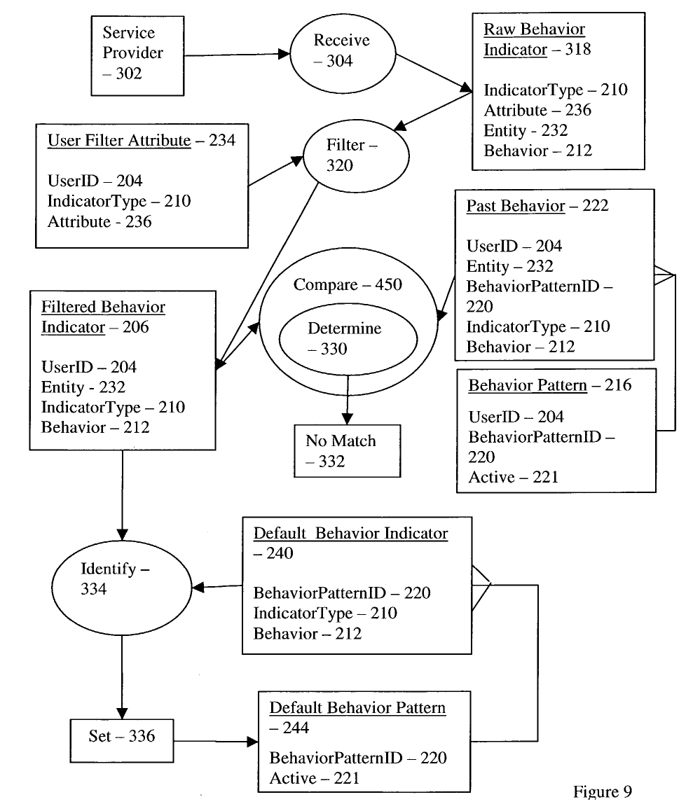

FIG. 9 is a data flow diagram illustrating a method of providing life support services to a user. The method of FIG. 9 includes receiving (304) a plurality of disparate behavior indicators. As discussed above with reference to FIG. 3, the disparate behavior indicators are represented in data structures as records such as, for example, the raw behavior indicator records (318) of FIG. 9. The term ‘raw’ means that the raw behavior indicators are not filtered for a user and that the raw behavior indicators are in a variety of data formats depending upon their types and upon the service providers where they originate. The term ‘disparate’ behavior indicators (318) means that the raw behavior indicator records (318) represent behavior indicators of different types, such as for example, behavior indicators (318) representing credit card purchases, GPS locations, computer logon times, or any other type of behavior indicator that will occur to those of skill in the art.

The method of FIG. 9 includes filtering (320) the behavior indicators (318) for a user in dependence upon user filter attributes, thereby producing filtered behavior indicators (206). As discussed above with reference to FIG. 3, filtering (320) the behavior indicators for a user in dependence upon user filter attributes typically includes converting raw behavior indicators into a predetermined internal data structure and comparing the converted raw behavior indicator record (318) with user filter attribute records (234).

The method of FIG. 9 includes identifying (334) a set (336) of default behavior indicators that match the filtered behavior indicators (206), wherein the set (336) of default behavior indicators identify a default behavior pattern (244). The term “default behavior indicator” is used to describe behavior indicators that are not behavior indicators generated as a result of a user's behavior, but are generic behavior indicators representing behaviors common to many users. A set (336) of default behavior indicators define a default behavior pattern. For example, a set (336) of default behavior indictors containing location coordinates for the city of Los Angeles may identify a default behavior pattern of being located in the city of Los Angeles. When a user's filtered behavior indicators match the default behavior indicators containing locations for the city of Los Angeles, a default behavior pattern that the user is in the city of Los Angeles is identified—even if the user has never before been to Los Angeles.

Default behavior indicators are represented in data structures as records such as, for example, the default behavior indicator records (240) of FIG. 9. The exemplary default behavior indicator records of FIG. 9 include a BehaviorPatternID field (220) identifying the default behavior pattern defined by the set (336) of default behavior indicators. The default behavior indicator records (240) include an IndicatorType field (210). The IndicatorType field (210) identifies a default behavior indicator by type such as credit card transaction, location, or any other default behavior indicator that will occur to those of skill in the art. The default behavior indicator records (240) include a behavior filed (212). The behavior field (212) represents a value for a default behavior indicator such as credit card purchase price, longitude and latitude coordinates, or any other value of a default behavior indicator that will occur to those of skill in the art. The default behavior indicator records (240) of FIG. 9 do not include the UserID field (204) and an Entity field (232) found in past behavior records (222), because default behavior indicators are generic to many users, not specific to any one user.

Default behavior patterns are represented in data structures as records such as, for example, the default behavior pattern record (244) of FIG. 9. The exemplary default behavior pattern records (244) include a BehaviorPatternID field (240) identifying the default behavior pattern. The default behavior pattern records (244) of FIG. 9 also include an active field (221). The active field is a Boolean indicator identifying if the default behavior pattern (244) is active. The default behavior patterns (244) are generic to many users, and therefore, do not include the UserID field (204) found in behavior pattern records (216) for a user.

A set (336) of default behavior indicators (240) define a default behavior pattern in a similar way that a set of past behavior records (222) define a behavior pattern (216) for a user. That is, a set (336) of default behavior indictor records (240) defining a default pattern are related many-to-one to the default behavior pattern record (244) through a BehaviorPatternID field (220) used as a foreign key.

In many examples of methods of providing life support services according to the present invention, default behavior patterns coexist with behavior patterns for a user. When the filtered behavior indicators (206) match a set (336) of default behavior indicators (240) identifying a default behavior pattern (244) and the filtered behavior indicators (206) match a set of past behavior records (222) identifying a behavior pattern (216) for the user both a default behavior pattern (244) and a behavior pattern for the user (216) are identified.

As an aid to explanation, consider the following example. A user frequently shops at the Galleria in Houston, Texas. When the user arrives at the Galleria, filtered behavior indicators containing locations for the user's car match a set of past behaviors for the user. A behavior pattern for the user is identified. The filtered behavior indicators may also match a set of default behavior indicators also containing locations of the Galleria. A default behavior pattern is also identified. An action may be taken in dependence upon both the identified behavior pattern for the user and the identified default behavior pattern. An action taken in dependence upon the user's behavior pattern of shopping at the Galleria is downloading, to the user's PDA, a list of stores in the shopping center having sales. Examples of default actions taken in dependence upon the default behavior pattern are contacting the user and informing the user of new stores at the Galleria, or contacting the user and informing the user about parking garages that are full and parking garages having available parking. As the previous example demonstrates, default behavior patterns may advantageously supplement behavior patterns for user.

Although pertinent default behavior patterns and behavior patterns for a user can coexist, it is an advantage of methods of providing life support services to a user according to embodiments of the present invention, however, to identify and helpfully act upon default behavior patterns even when such a method of providing life support services, at any particular point in time, is unable to identify a behavior pattern for a user based upon the particular user's past behavior. The method of FIG. 9, for example, includes comparing (450) the filtered behavior indicators (206) with a plurality of past behavior records (222) for a user. In the method of FIG. 9, comparing (450) the past behavior records (222) and the filtered behavior indictors (206) includes determining (330) that the past behavior records (222) do not match (332) the filtered behavior indicators (206). In this example, because the past behavior records (222) do not match the filtered behavior indicators, no behavior pattern for the user is identified.

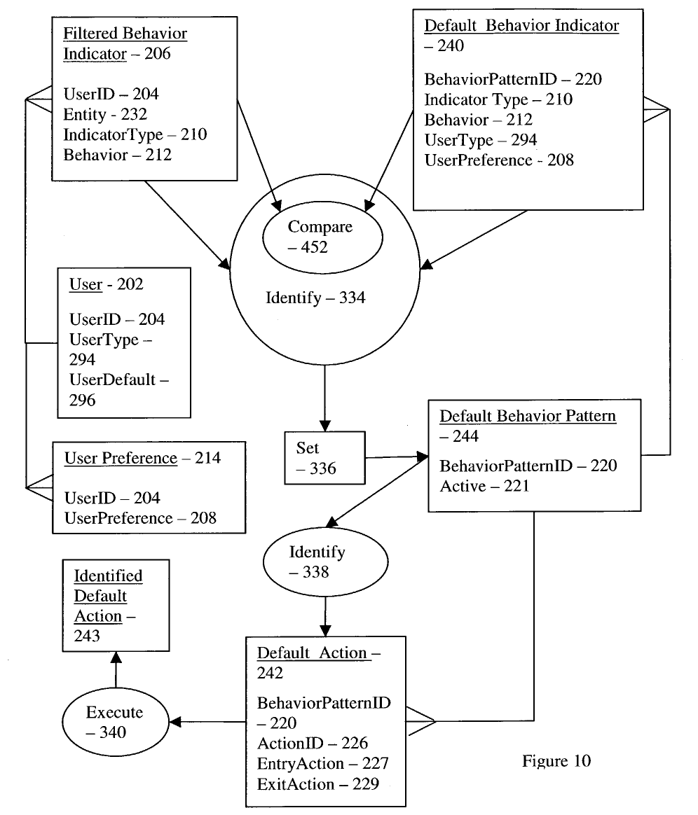

FIG. 10 is a data flow diagram illustrating in more detail the process of identifying (334) a set (336) of default behavior indicators (240) that match filtered behavior indicators (206). Identifying (334) a set (336) of default behavior indicators that match filtered behavior indicators (206) includes comparing (452) filtered behavior indicators (206) with default behavior indicators (240). When a set (336) of default behavior indicators (240) match the filtered behavior indicators (206) a default behavior pattern (244) is identified by locating a default behavior pattern record (244) related to the set (336) of default behavior indicators (240) through the BehaviorPatternID field (220) used as a foreign key.

It is useful by way of explanation to note that identifying (334) a set (336) of default behavior indicators (240) that match filtered behavior indicators (206) does not require that the default behavior indicators (240) perfectly match the filtered behavior indictors to identify a default pattern (244). The degree to which the filtered behavior indicator records (206) must match the default behavior indicator records (240) to identify a default behavior pattern (244) depends on various factors such as the tolerances of the methods used for comparing (452) the filtered behavior indicators (206) and the default behavior indicators (240), the type of information contained in the filtered behavior indicator records (206) and the default behavior records (240), the accuracy and precision used in generating the filtered behavior indicator records (206) and the default behavior indicator records (240), user specified conditions, and other factors that will occur to those skilled in the art.

While default behavior patterns are generic to many users, the default behavior patterns and actions taken in dependence upon the default behavior patterns may be tailored to certain types of users. For example, users having the same occupation may engage in similar behavior patterns. Furthermore, default behavior patterns for one type of user may not appropriately reflect a default behavior pattern for another type of user. Consider the following example. Two users are subscribers to life support services according to the present invention. One user is buyer for a large company. The other user is a fireman. The buyer may incur large credit card transactions as a part of the buyer's occupation. The credit card purchases may be used to identify default behavior patterns for a buyer. However, the same kind of credit card purchases may identify a behavior pattern for a stolen credit card for the fireman.

To more specifically define or tailor default behavior patterns, the default behavior indicators (240) and the user records (202) of FIG. 10 include a UserType (294) field. The UserType field (294) is a type code identifying the type of user. An example of a type code for a user is an occupation code identifying the occupation of the user. In some examples according to the method of the present invention, identifying (334) a set (336) of default behavior indicators (240) that match the filtered behavior indicators (206) includes locating a user record (202) related to the filtered behavior indicators (206) through the UseriD field (204) used as foreign key, and comparing the UserType (294) field of the user record (202) with the UserType field (294) of the default behavior indicators.

Users having similar interests may also engage in similar behavior patterns. Therefore, some typical embodiments of a method of providing life support services to a user include identifying a default behavior pattern in dependence upon user preferences. User preferences include a user's interests, a user's association with a particular association or any other user preference that will occur to those of ordinary skill in the art. An example of a user preference is an appreciation for blues music. User's preferences are represented as data structures such as the user preference records (214) of FIG. 10. Because a user may have more than one preference, the user preference records (214) are related many-to-one to the user records (202) through the userld (204) field used as a foreign key. The user preference records (214) include a user preference field (208), including for example, type codes identifying various user preferences.

Identifying a default behavior pattern in dependence upon user preferences includes comparing the user preference field of the default behavior indictor records (240) with the user preference field (208) of the user preference record (214) for a user. Typically, comparing the user preference field of the default behavior indicator record (240) with the user preference field (208) of the user preference record (214) includes locating a user preference record (214) through a user record (202) related to both the filtered behavior indictor records (206) and the user preferences records (214) through the UseriD field (204) used as a foreign key.

Consider the following example. Default behavior indicators include locations for the city of Chicago. The user's preference record (214) includes a type code identifying the user as a fan of blues music. When the user is in Chicago, the users filtered behavior indicators match a set of default behavior indicators defining a default behavior pattern of a blues fan in Chicago. An example of a default action to be taken in dependence upon the identified behavior pattern may be downloading information about Chicago blues clubs to the users PDA.

In some examples of methods of providing life support services to a user, identifying default behavior patterns are a user option. That is, a user may not want default behavior patterns to be identified. In one example of providing life support services to a user, wherein identifying default behavior patterns is a user option, a UserDefault field (296) is included in the user records (202) as shown, for example, in the user records (202) of FIG. 10. The UserDefault field (296) is a Boolean indication whether default behavior patterns are to be identified for the user. The UserDefault field (296) in the user record (202) is set true or false depending on whether default behavior patterns are to be identified for the user. If an indication whether default behavior patterns are to be identified as life support for a particular user is set to ‘false,’ then methods of life support according to embodiments of the present invention typically do not attempt to identify default behavior patterns in providing life support for that user.

Returning briefly to the information handling systems of FIG. 1, an example of one way a user can set the UserDefault field in the user record to true or false, is by accessing the user record stored on an LSS server (104). Using a web browser installed on a computer (112) a user accesses a user record stored on an LSS Server (104) across an internet (118). In some typical embodiments, an LSS application (128) running on the LSS Server (104) may prompt the user to enter an instruction to set the UserDefault field in the user record true or false, receive the instruction from the user, and carry out setting the UserDefault field in the user record. In various exemplary embodiments, the UserDefault field is set true or false in any method that will occur to those of skill in the art.

The method of FIG. 10 also includes identifying (338) a default action (242) to be executed in dependence upon a default behavior pattern. The term ‘default action’ is used for clarity of explanation. The term ‘default action’ is used to mean an LSS action identified and executed in dependence upon a default behavior pattern. The default actions (242) operate in a similar manner as the actions (223) of the method of FIG. 3 in that default actions provide helpful life support for a user in dependence upon default behavior patterns. By way of further explanation, one distinction between the default actions of the method of FIG. 10 and the actions of the method of FIG. 3 is that the default actions of FIG. 10 are identified in dependence upon default behavior patterns and the actions of the method of FIG. 3 are identified in dependence upon behavior patterns for a user.

Default actions are represented in data structures as records such as the default action record (242) of FIG. 10. The default action records (242) also include an actionID (226) identifying the action to be taken in dependent upon the default action. The default action records (242) include a BehaviorfD field (220) identifying the default behavior pattern in whose dependence the default action identified by the ActionID field (226) is taken. Because more than one default action may be identified for a default behavior pattern, the default action records (216) are related many-to-one to the default behavior pattern records through the BehaviorPatternID field (220) used as a foreign key. The default action record (242) also includes an EntryAction field (227) and an ExitAction field (229) identifying the default action as an entry action, exit action, or both an entry action and exit action.

The method of FIG. 10 also includes executing (340) an identified default action (243). Executing (340) identified default actions according to the method of FIG. 10 is carried out in a similar manner as executing (316) the actions (223) of FIG. 3, that is, actions identified in dependence upon behavior patterns for users. More particularly, alternative exemplary methods of executing (340) a default action (243) include reading a filename for a computer program from an action record and executing the program, deploying an SBB component in a SLEE environment, deploying a service interface in a Parlay environment, obtaining from an action factory class a reference to a default action object and polymorphically calling a factory method in the default action object, or any other method of executing the identified default action as will occur to those of skill in the art.

### Identifying Abstract Behavior Patterns from Filtered Behavior Indicators

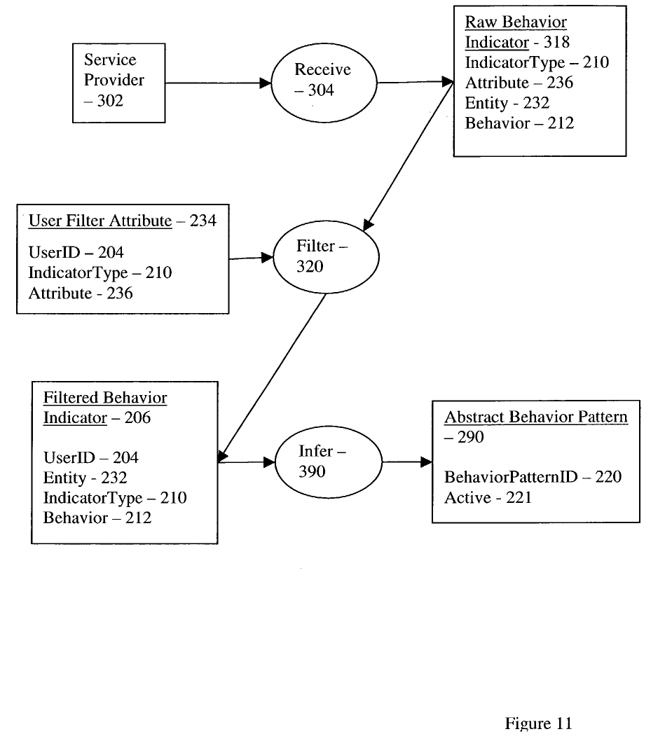

FIG. 11 is a data flow diagram illustrating a method of providing life support services to a user. The method of FIG. 11 includes receiving (304) a plurality of disparate behavior indicators. In the method of FIG. 11, disparate behavior indicators are implemented as the raw behavior indicator records (318) of FIG. 11. The term ‘raw’ means that the raw behavior indicators, such as the raw behavior indicator records (318), are not filtered for a user and that the raw behavior indicators are in a variety of data formats depending upon their types and upon the service providers where they originate. The term ‘disparate’ behavior indicators (318) means that the raw behavior indicator records (318) represent behavior indicators of different types, such as for example, behavior indicators (318) representing credit card purchases, GPS locations, computer logon times, or any other type of behavior indicator that will occur to those of skill in the art.

The method of FIG. 11 includes filtering (320) the behavior indicators (318) for a user in dependence upon user filter attributes, thereby producing filtered behavior indicators (206). As described in more detail above, filtering (320) the behavior indicators for a user in dependence upon user filter attributes typically includes converting raw behavior indicators into a predetermined internal data structure and comparing the converted raw behavior indicator record (318) with user filter attribute records (234).

The method of FIG. 11 includes inferring (390) an abstract behavior pattern in dependence upon the filtered behavior indicators (206). An ‘abstract behavior’ pattern is a behavior pattern that is inferred from the context of the user's filtered behavior indicators without reference to the user's past behaviors. For example, if a series of filtered behavior indicators for a user indicates that the user's car has taken 16 right turns in a very short period of time, and the 16 right turns correspond to circling a particular block four times, an abstract behavior pattern may be inferred that the user is lost. This abstract behavior pattern may be inferred from the filtered behavior indicators themselves without reference to the user's past behaviors or default behavior indicators.

Abstract behavior patterns according to some example methods of the present invention are represented in data structures as records such as the abstract behavior pattern record (290) of FIG. 11. The abstract behavior pattern record of FIG. 11 includes a BehaviorPatternID (220) identifying the abstract behavior pattern. The abstract behavior pattern record of (290) also includes an active field (221). The active field (221) is a Boolean indicator identifying whether the abstract behavior pattern is currently active, that is, currently being used to identify actions to take for user life support.

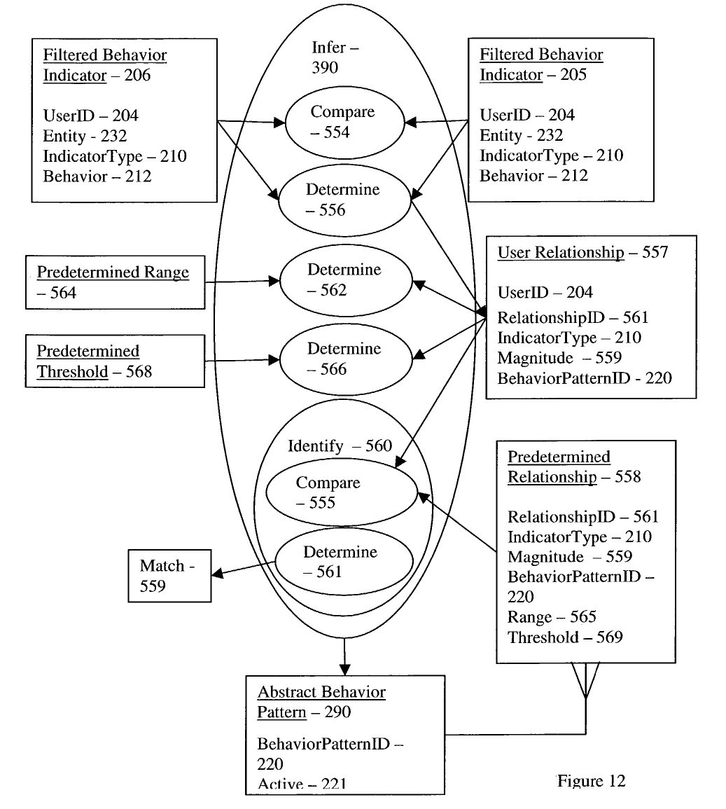

FIG. 12 is a data flow diagram illustrating inferring (390) an abstract behavior pattern (290) in dependence upon filtered behavior indicators (206). Inferring (390) an abstract behavior pattern (290) in dependence upon filtered behavior indicators (206) includes comparing (554) a first filtered behavior indicator (206) with a second filtered behavior indicator (205). Inferring (390) an abstract behavior pattern (290) in dependence upon filtered behavior indicators according to the method of FIG. 12 also includes determining (556) a relationship (557) between the first filtered behavior indicator (206) and the second filtered behavior indicator (206).

The term relationship in this specification means a description of a particular behavior determined from at least two filtered behavior indicators. For example, a relationship between two filtered behavior indicators arriving sequentially in working cache for a user that include the same coordinates (indicating the same physical location), but differing time stamps (indicating that time has passed) may be ‘no movement.’

Examples of relationships determined from at least two filtered behavior indicators includes direction of travel, a turn, speeds of travel, or any other relationship that can be determined from the filtered behavior indicators as will occur to those of skill in the art.

Determining (556) a relationship (557) may include subtracting a first filtered behavior indicator from a second filtered behavior indicator, adding a first filtered behavior indicator to a second filtered behavior indicator, or using any other function or method to determine a relationship between a first filtered behavior indicator and a second filtered behavior indicator that will occur to those of skill in the art. For example, one way of determining a direction of travel from two coordinates identifying physical locations includes designating the first coordinate as the beginning of a line, designating the second coordinate as the endpoint of the line, and comparing the line with a coordinate system, or compass. In one example of providing life support services to a user according to the method of FIG. 12, determining (556) a relationship is carried out by an LSS application programmed to identify predefined relationships from filtered behavior indicators.

Determining a relationship between the first filtered behavior indicator and the second filtered behavior indicator also includes creating a user relationship record (557) representing the determined relationship having a predetermined internal data structure such as the structure of the user relationship record of FIG. 12. The user relationship records (557) are called ‘user’ relationship records because they represent relationships among the user's filtered behavior indicator records. The user relationship record (557) is a predetermined internal data structure designed for specific relationships between the filtered behavior indicators. For example, user relationship records representing a direction of travel may have one data structure and user relationship records no movement may have another. The user relationship record of FIG. 12 is a generalized record provided for purposes of explanation of the method of providing life support services to a user.

In many embodiments of the present invention, determining user relationships among filtered behavior indicators is carried out algorithmically. That is, for example, in terms of computer program coding, determining user relationships among filtered behavior indicators is carried out by use of switch statements or sequences of if-then-else statement implementing logic similar to that illustrated for example as follows:

If a first filtered behavior indicator represents location 1

If a second filtered behavior indicator represents location 2

If location 1 is not equal to location 2

Then make a user relationship record representing the relationship of ‘in motion.’

Or

If a first filtered behavior indicator represents location L1 at time t1

If a second filtered behavior indicator represents location L2 at time t2

If location L1 is not equal to location L2

Then

Calculate speed S and direction D

and

make a user relationship record representing the relationship of ‘in motion’ with magnitude fields for speed set to S and direction set to D

Inferring (390) an abstract behavior pattern (290) is not limited to comparing only two filtered behavior indicators. In fact, in many examples of providing life support services to a user, inferring (390) an abstract behavior pattern (290) includes comparing many filtered behavior indicators (206) and determining many relationships (557) among the filtered behavior indicators (206). As an example of comparing more than two filtered behavior indicators, consider the following example of algorithmic logic for determining the user relationship ‘turned right.’

If a first filtered behavior indicator represents location L1 at time t1

If a second filtered behavior indicator represents location L2 at time t2

If a third filtered behavior indicator represents location L3 at time t3

If L1<>L2<>L3

Then

Calculate direction D1 from L1 and L2

Calculate direction D2 from L2 and L3

If D1−D2 is approximately 90 degrees

Then

make a user relationship record representing the relationship of ‘turned right.’

The user relationship record (557) of FIG. 12 includes a userId field (204) identifying the user for whom abstract behavior patterns are to be identified. The user relationship record (557) of FIG. 12 includes a relationshipID field (561) identifying the determined relationship. The user relationship record (557) of FIG. 12 also includes an IndicatorType field (210) identifying the type of filtered behavior indicators compared to determine the relationship. The user relationship record (557) also includes a magnitude field (559) identifying the magnitude of the relationship.

Continuing with the location examples, if the relationship record (557) has a relationshipID identifying the relationship of ‘traveling north,’ a magnitude field (559) may include the speed of travel. A relationship record can have more than one magnitude field. If a relationship record represents a relationship of ‘moving,’ magnitude fields can indicate direction and speed.

In the method of FIG. 12, inferring (390) an abstract behavior pattern (290) includes identifying (560) an abstract behavior pattern (244) in dependence upon the determined relationship. Identifying (560) an abstract behavior pattern (244) in dependence upon the determined relationship includes comparing (555) the user relationship records (557) with a set of predetermined relationships (558) and determining (561) that the set of predetermined relationships (558) match the user relationships (107). Identifying (560) an abstract behavior pattern (290) also includes locating an abstract behavior pattern record (290) related to the predetermined relationship records that match the user relationship records (557).

The predetermined relationships (558) are predefined relationships a set of which define an abstract behavior pattern. For example, for predefined relationship records for turning right may define an abstract behavior pattern of traveling in a square. The relationships may be generic relationships identified from other user's filtered behavior indicators, or calculated relationships which are not derived from any user's filtered behavior indicators. The predetermined relationships (558) are represented in data structures as records such as the predetermined relationship records (558) of FIG. 12. A set of predetermined relationships (558) define an abstract behavior pattern (290), and therefore, a set of predetermined relationship records are related many to one to the abstract behavior pattern record through the BehaviorPatternID field (220) used as a foreign key.

Consider the following example. A plurality of filtered behavior indicators containing coordinates provided by a user's GPS watch indicate the user is on Michigan Avenue in Chicago. The filtered behavior indicators also include credit card transactions from stores on Michigan Avenue in Chicago. The filtered behavior indictors are compared and a relationship is determined for moving along the perimeter of the block in Chicago. A relationship for moving toward the center of a block is determined. Another relationship of making multiple purchases from the a store is determined. These determined relationships match a set of predefined relationships identifying an abstract behavior pattern for shopping.

Consider another example, filtered behavior indicators for a user may indicate that the user is logged onto a network at work, the user has swiped an ID badge entering the user's office building, and the user is sitting at his desk. However, the filtered behavior indicators may indicate that the user' car is traveling away from the user at a very high speed. The filtered behavior indicators are compared and a set of relationships among the filtered behavior indicators are determined. The relationships between the user and the car matched with predetermined relationships for car theft identify an abstract behavior pattern that the user's car has been stolen.

Although the example above have discussed determining user relationships by comparing filtered behavior indicators, in fact, determining user relationships can also be carried out by comparing user relationships themselves. In this disclosure, user relationships determined by comparing user relationships are referred to as ‘secondary.’ That is, inferring an abstract behavior pattern in many embodiments includes determining secondary user relationships. Secondary user relationships are relationships among the user relationships that were in turn determined from the filtered behavior indicators. For example, a secondary user relationship of moving in a circle may be determined from four right turn user relationships determined from the filtered behavior indictors. Moving in a circle may be a relationship which in part defines a abstract behavior pattern.

Determining secondary user relationships by comparing user relationships can advantageously simplify the overall process of determining user relationships. More particularly, consider how the exemplary algorithmic logic set forth above for ‘turned right’ is simplified by applying comparing user relationships and treating ‘turned right’ as a secondary user relationship:

If a first user relationship represents ‘in motion’ with magnitude D1

If a second user relationship represents ‘in motion’ with magnitude D2

if D1−D2 is approximately 90 degrees

then make a user relationship record representing the relationship of ‘turned right.’

In the method of FIG. 12, inferring (390) an abstract behavior pattern (290) includes determining (562) that the relationship is within a predetermined range (564). In some typical embodiments, determining (562) that the determined relationship is within a predetermined range includes comparing the magnitude of a user relationship record (557) with the predetermined range. Determining (562) that the magnitude of the relationship is within a predetermined range provides guidelines for inferring an abstract behavior pattern.

Consider an example with a user and the user's child at an amusement park. Both the child and user are wearing an RFID or GPS wristbands resulting in the generation of filtered behavior indicators including the location of the user and the child. The filtered behavior indicators are compared to determine user relationships. One user relationship is the separation between the user and the child represented by a user relationship record having a magnitude field representing the size of the separation between user and child. When the separation is within a first range, an abstract behavior pattern that the child and parent are together is inferred. When the separation between the child and user is not within the range an abstract behavior pattern may be inferred that the child and user have become separated. An action may be executed in dependence upon the inferring that that the child and user have become separated. One example of such an action is contacting the user and informing the user of the child's location.

One way of inferring an abstract behavior pattern using a predetermined range, includes having a range field (565) in the predetermined relationship records (558) that define abstract behavior patterns (290) such as the predetermined relationship records (558) of FIG. 12. The range field (565) includes a type code identifying the predetermined range (564) that the magnitude (559) of the predetermined relationship is within. Alternatively, the range field (565) may be a Boolean indication that the magnitude is within a predetermined range.

In the method of FIG. 12, inferring (390) an abstract behavior pattern (290) includes determining (566) whether the determined relationship (557) exceeds a predetermined threshold (568). In some typical embodiments, determining (562) that the relationship exceeds a predetermined threshold includes comparing the magnitude of a user relationship record (557) with a predetermined threshold. A predetermined threshold provides an additional guideline for inferring abstract behavior patterns.

Returning to the previous example of the child and user at an amusement park. The filtered behavior indicator are compare and a relationship that the user and child are separated is determined. When the separation between the user and the exceeds a predetermined threshold, a behavior pattern that the child is lost may be inferred. An action such as contacting emergency services may be executed in dependence upon inferring that the child is lost.

One way of inferring an abstract behavior pattern using a predetermined threshold, includes having a threshold field (569) in the predetermined relationship records (558) that define abstract behavior patterns (290) such as the predetermined relationship records (558) of FIG. 12. The threshold field (569) includes a type code identifying a predetermined threshold (566) compared with the magnitude (559) and whether the magnitude exceeds the threshold. Alternatively, the threshold field (569) may be a Boolean indication that the magnitude exceeds a predetermined threshold.

Comparing the magnitude with a predefined threshold can be used in conjunction with determining if the magnitude is within a predefined range. Still continuing with the example of a user and a child at an amusement park, if the separation between the child and the user is within a range, an abstract behavior pattern that the user and child are separated, but not dangerously separated may be inferred. An action executed in dependence upon this abstract behavior pattern may include calling the user on the user's mobile phone and informing the user of the child's location. If the separation is not within the second range and is beyond a predefined threshold, an abstract behavior pattern may be inferred that the child is in danger. An action in response to this abstract behavior pattern may be notifying emergency services of the child's location.

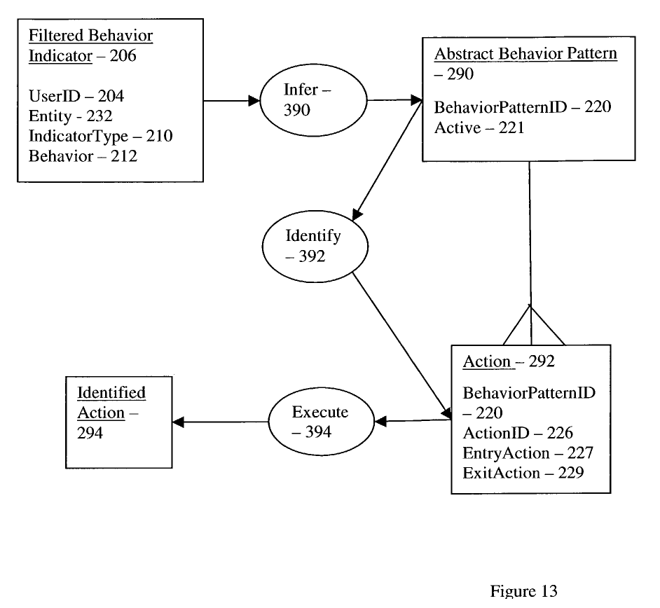

FIG. 13 is a data flow diagram illustrating identifying (392) an action (292) to be taken in dependence upon the abstract behavior pattern (290). The actions to be taken in dependence upon an abstract behavior pattern are identified and operate in the same manner as the actions (223) of the method of FIG. 3, except that the actions of the method of FIG. 13 are identified in dependence upon abstract behavior patterns and the actions of the method of FIG. 3 are identified in dependence upon behavior patterns for a user.

Actions to be taken in dependence upon an abstract behavior pattern (290) are represented as records such as the action record (292) of FIG. 13. The action records of FIG. 13 includes a BehaviorPatternID field (220) identifying the abstract behavior pattern in whose dependence the action is taken. The action records of FIG. 13 also include an ActionID field (226) identifying the action to be taken in dependence upon the abstract behavior pattern. Because more than one action may be taken in dependence upon an abstract behavior pattern, the action records (292) are related many to one to the abstract behavior pattern records through the BehaviorPatternID (220) field used as a foreign key. The action records include and EntryAction field (227) and an ExitAction field (229) identifying if the action record represents an entry action an exit action or both an entry action and an exit action. Identifying (392) an action to be taken in dependence upon the abstract behavior pattern includes locating the action records (292) related to the abstract behavior pattern record and reading the actionID (226) from the action records (292).

The method of FIG. 10 also includes executing (394) an identified action (294). Executing (394) identified actions according to the method of FIG. 13 is carried out in a similar manner as executing (316) the actions (223) of FIG. 3, that is, actions identified in dependence upon behavior patterns for users. More particularly, alternative exemplary methods of executing (394) an action (243) include reading a filename for a computer program from an action record and executing the program, deploying an SBB component in a SLEE environment, deploying a service interface in a Parlay environment, obtaining from an action factory class a reference to an action object and polymorphically calling a factory method in the action object, or any other method of executing an identified action as will occur to those of skill in the art.

### Creation of New Behavior Patterns for a User

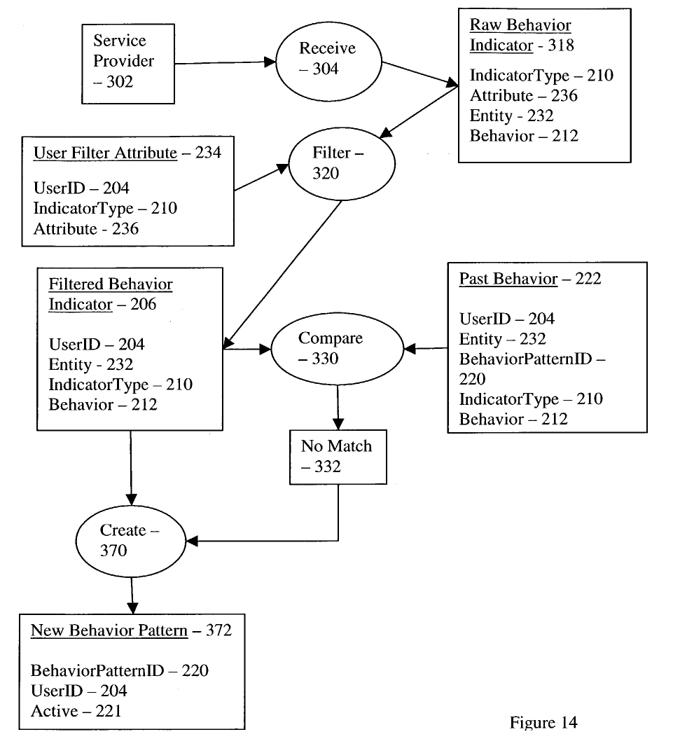

FIG. 14 is a data flow diagram illustrating a method of providing life support services to a user. The method of FIG. 14 includes receiving (304) a plurality of disparate behavior indicators. In the method of FIG. 14, disparate behavior indicators are implemented as the raw behavior indicator records (318) of FIG. 14. The term ‘raw’ means that the raw behavior indicators, such as the raw behavior indicator records (318), are not filtered for a user and that the raw behavior indicators are in a variety of data formats depending upon their types and upon the service providers where they originate. The term ‘disparate’ behavior indicators (318) means that the raw behavior indicator records (318) represent behavior indicators of different types, such as for example, behavior indicators (318) representing credit card purchases, GPS locations, computer logon times, or any other type of behavior indicator that will occur to those of skill in the art.

The method of FIG. 14 includes filtering (320) the behavior indicators (318) for a user in dependence upon user filter attributes, thereby producing filtered behavior indicators (206). As described in more detail above, filtering (320) the behavior indicators for a user in dependence upon user filter attributes typically includes converting raw behavior indicators into a predetermined internal data structure and comparing the converted raw behavior indicator record (318) with user filter attribute records (234).

The method of FIG. 14 includes comparing (330) the filtered behavior indicators (206) with a plurality of past behavior records (222) for a user, wherein comparing (330) the filtered behavior indicators (206) with a plurality of past behavior records (222) results in a determination (332) that no set of past behavior records (222) identifying a behavior pattern (216) for a user matches the filtered behavior indicators (206). When the filtered behavior indicators (206) for the user do not match (332) a set of past behavior records (222) the user is said to be acting ‘out of pattern’ and no behavior pattern for the user is identified.

The method of FIG. 14 includes creating (370) a new behavior pattern for the user. The phrase ‘new behavior pattern’ is used for clarity of explanation. When the new behavior pattern is created, the new behavior pattern is implemented and operates in the same way as behavior patterns for the user generally, as described above in the discussion of the method according to FIG. 3. The new behavior patterns are represented in data structures as records such as, for example, the new behavior pattern records (372) of FIG. 14. The new behavior pattern records (372) include a BehaviorPatternID (220) identifying the new behavior pattern. The new behavior pattern records (372) of FIG. 14 includes a UserID field (204) identifying the user. The new behavior pattern also includes an active field (221). The active field (221) is a Boolean indicator identifying the state of the new behavior pattern record. When the new behavior pattern is created, the active field is set false. When the new behavior pattern is subsequently identified, the active field is set true indicating the state of the new behavior pattern as active, that is, as currently in use to identify and carry out helpful life support actions for a user.

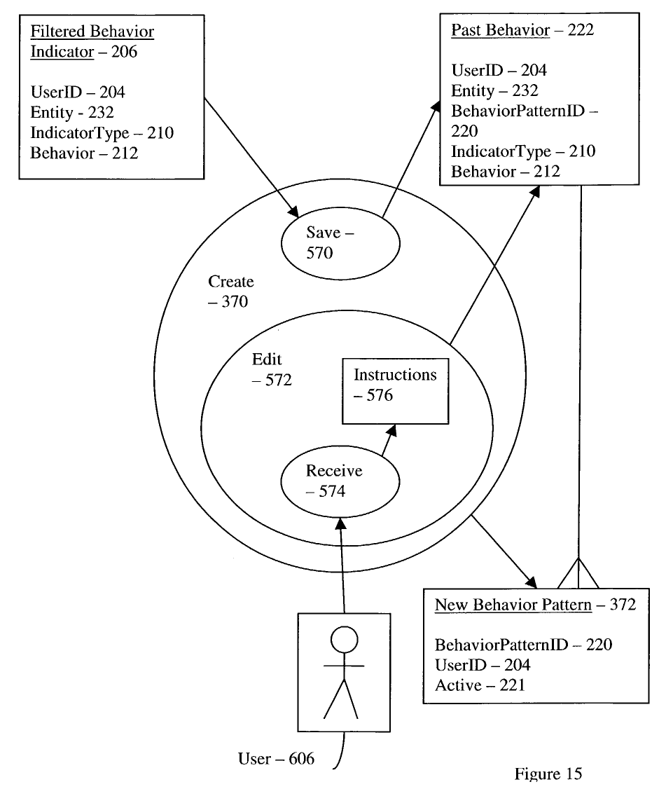

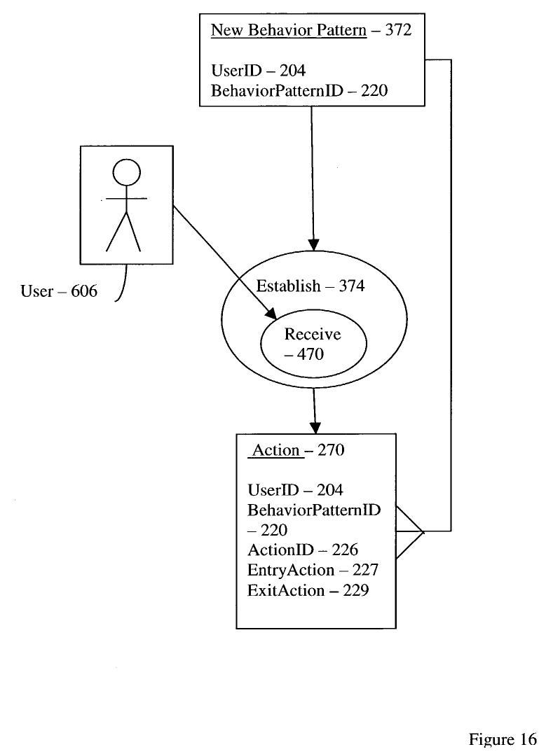

FIG. 15 is a data flow diagram illustrating a method of creating (370) a new behavior pattern (372) for a user. The method of FIG. 16 includes saving (570) the filtered behavior indicators (206) as past behavior records (222). Saving the filtered behavior indicators (206) as a past behaviors record (222) typically includes adding an additional BehaviorPatternID field (220) having the same BehaviorPatternID as the new behavior pattern record (372). The set of filtered behavior indicators saved as past behaviors are related to the new behavior pattern record through the BehaivorPatternID field used as a foreign key.

One way of creating a new behavior pattern for a user includes saving all of the filtered behavior indicators in working cache as past behaviors when it is determined that the user is out of pattern. All of the filtered behavior indicators in working cache are treated as representing a new behavior pattern for the user and saved as past behaviors with a default pattern name.

By saving all the filtered behavior indicators as past behaviors when the user is out of pattern, however, filtered behavior indicators that are unnecessary or undesirable may be saved as past behavior records. The method of FIG. 15 therefore includes editing (572) the past behavior records (222). Editing the past behavior records (222) that identify a new behavior pattern includes deleting, changing, or otherwise augmenting the past behavior records to more accurately identify the new behavior pattern.

As an aid to explanation, consider the following example. Filtered behavior indicators for a user include locations of the user's car, telephone numbers the user is calling on the user's mobile telephone. The filtered behavior indicators do not match any existing behavior pattern for the user, because the user is traveling to a new client's office—a new location for the user. Therefore, the user is out of pattern. All of the filtered behavior indicators are initially saved as a new behavior pattern for the user. The filtered behavior indicators identifying the car's location are relevant to a new behavior pattern of traveling to the new client's office. However, the telephone calls are unrelated to the new behavior pattern of traveling to the user's new client. Therefore, the user may edit the past behaviors identifying the new behavior pattern of traveling to the client's office by deleting the past behaviors that are unnecessary in defining the new behavior pattern.

Continuing with the example of the last paragraph, if the user is traveling to a new client's office and the location records indicate route the user took to the client's office. The user may want the new behavior pattern to include different routes to the new client's office. One way of including different routes to the new client's office is to delete some of the filtered behavior to make the behavior pattern of traveling to the new client's office broader. The behavior pattern is broader, because fewer filtered behavior indicators are necessary to identify the new behavior pattern and consequently, the new behavior pattern of traveling to the new client's office may be identified when the user takes multiple routes to the new client's office.

The method of FIG. 15, editing (572) the past behavior records (222) includes receiving (574) editing instructions (576) from the user (606). By receiving editing instructions from a user, the method of FIG. 15 advantageously allows the user to edit the past behaviors to more particularly define the new behavior pattern.

In some typical examples of methods according to the present invention, editing (572) the past behaviors is carried out by an LSS application (128) running on an LSS server (104) such as the LSS Server (104) and LSS application (128) of FIG. 1. The user may access past behaviors stored on the LSS server (104) through and internet (118) using a web browser installed on a computer (112). The LSS application (128) prompts the user step-by-step to input editing instructions and the LSS application (128) carries out editing the past behaviors.

Another way an LSS application (128) may receive editing instructions from a user is across a circuit switched network (110) such as the telephone network (112) of FIG. 1. An LSS application (128) may prompt the user to input editing instructions. The user may input the editing instructions using a telephone key pad. The LSS application (128) may receive the editing instructions as DTMF signals and carry out editing the past behaviors in accordance with the editing instructions received as DTMF signals.

In still further examples of methods of providing life support services to a user in accordance with the present invention, create new behavior patterns by creating new past behaviors without actually engaging in behavior that results in the generation of behavior indicators. The past behaviors are directly created as records. Some embodiments, for example, provide data entry screens for users to edit past behavior records, including controls labeled, for example, ‘add’ or ‘new’ that leads to an editing screen depicting a blank past behavior record template for the user to fill in.

One way of creating past behaviors a records includes accessing a past behavior record template. An LSS application (128) running on an LSS Server (104) such as the LSS application (128) illustrated in FIG. 1 may provide a past behavior record template and prompt the user to input fields such as UserID, Entity, BehaviorPatternID, IndicatorType, and Behavior fields. The LSS application carries out creating the past behavior records. In this way, behavior patterns for a user may be generated without the user having to actually engage in the behavior pattern.

FIG. 16 is a data flow diagram illustrating a method of establishing (374) an action (270) associated with the new behavior pattern (372) for the user. An action associated with the new behavior pattern (372) is an action to be taken in dependence upon the new behavior pattern. That is, when the new behavior pattern is subsequently identified because the filtered behavior indicators for a user match the past behaviors related to the new behavior indicator, the action associated with the new behavior pattern is identified and executed. The actions associated with the new behavior pattern operate in the same manner as actions taken in dependence upon behavior patterns for a user discussed with reference to FIG. 3.

Actions associated with the new behavior pattern are represented in data structures as records such as the action records (270) of FIG. 16. The action records include a UserID field (204) identifying the user. The action records of FIG. 16 also include a BehaviorPatternID field (220) identifying the new behavior pattern for the user associated with the action record (270). Because more than one action may be associated with the new behavior pattern, the action records (270) are related many to one through the UserID field (204) and the BehaviorPatternID field (220) used as a composite foreign key.

The action records of FIG. 16 also include an actionID field (226). The actionID field (226) identifies the action associated with a new behavior pattern (372) for the user. In one example, the actionID field (226) may include a filename identifying a file that when run executes the action associated with the new behavior pattern. The action records (270) also include an EntryAction field (227) and an ExitAction field (229) identifying the action as an entry action, an exit action, or both an entry action and exit action.

One way of establishing an action (270) associated with the new behavior pattern (372) for the user includes receiving (470) the action (270) from a user. In some typical embodiments, receiving (470) the action (270) from a user (606) includes receiving a selection of a particular action from a list of available actions disclosed to the user through a pull down menu on a graphical user interface on a personal computer. The selected action is then associated with the new behavior pattern as an action to be taken in dependence upon the new behavior pattern, by creating an action record corresponding to the selected action. As a result, when the new behavior pattern is subsequently identified, the action associated with the new behavior pattern is identified and executed.

Establishing an action (270) associated with the new behavior pattern (372) may be carried out with an LSS application (128) running on an LSS server (104) such as, for example, the LSS application (128) running on an LSS server (104) as shown on FIG. 1. A user may access the new behavior pattern on the LSS Server (104) through an internet (118) using a web browser installed on a computer (122). The LSS application (128) may provide a selection of available actions. The user may then associate the available actions with the new behavior pattern by selecting an action. The user may select the action as an entry action, an exit action, or both an entry and exit action. In response to the user's selection of an available action, the LSS application (128) may carry out creating an action record representing the action associated with the new behavior pattern. The action record is related to the behavior pattern record through the BehaviorPatternID field (220) used as a foreign key. The action record is created with a UserID field identifying the user.

### Generating Behavior Patterns for a User from Historical Behavior Indicators

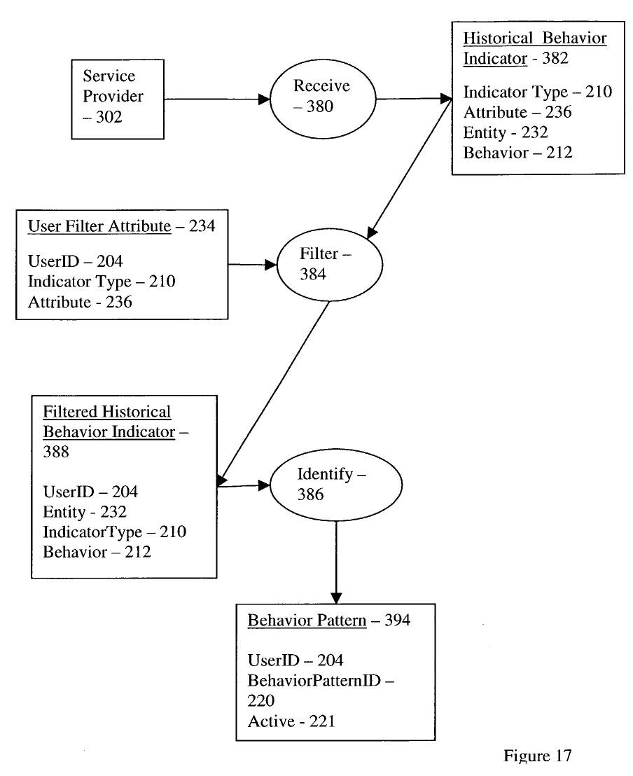

FIG. 17 is a data flow diagram illustrating a method of providing life support services to a user. The method comprises receiving (380) a plurality of disparate historical behavior indicators. The disparate historical behavior indicators are represented in data structures as records such as the historical behavior indicator records (382) of FIG. 17. The term ‘historical’ means that the historical behavior indicators are behavior indicators that have existed with service providers or with the user for some period of time prior to subscription or initiation with the methods of providing life support services of the present invention. The term ‘disparate’ means that the historical behavior indicator records (382) represent behavior indicators of different types, such as for example, behavior indicators representing credit card purchases, GPS locations, computer log on times, or any other type of behavior indicator that will occur to those of skill in the art.

Historical behavior indicators are typically a collection of behavior indicators from which behavior patterns for a user are not yet identified. In typical embodiments of the present invention, historical behavior indicators are created as a result of a user's behavior prior to the user's subscription or initiation with methods of providing life support services, or prior to using the historical behavior indicators to identify behavior patterns for a user. In some example embodiments, historical behavior indicators (382) include behavior indicators that have existed for days, weeks, months, or even years before initiating life support services for a user. In other examples, the historical behavior indicators include behavior indicators produced after subscription or initiation with life support services, but before using the historical behavior indicators to identify behavior patterns for a user. An example of historical behavior indicators (382) may include days, months, or years of credit card transaction data maintained in electronic form, location information tracked by mobile phones or GPS systems, or any other historical behavior indicators that will occur to those of skill in the art.

The method of FIG. 17 includes filtering (384) the historical behavior indicators (382) for a user in dependence upon user filter attributes (234). Filtering (384) the historical behavior indicators (382) is carried out in the same manner as filtering raw behavior indicators discussed above with reference to FIG. 3. Filtering (384) the historical behavior indicators (382) for a user in dependence upon user filter attributes (234) typically includes converting the historical behavior indicators (382) into a predetermined internal data structure and comparing the converted historical behavior indicator record (382) with user filter attribute records (234). Filtering (384) the historical behavior indicators (382) for user results in a collection of filtered historical behavior indicators which are behavior indicators associated with a user and converted into a predetermined internal data structure.

The method of FIG. 17 includes identifying (386) a behavior pattern for the user in dependence upon the filtered historical behavior indicators (388). Behavior patterns for a user are represented in data structures as records such as the behavior pattern record (394) of FIG. 17. The behavior pattern record (394) includes a UserID field (204) identifying the user. The behavior pattern record (394) includes a BehaviorPatternID field identifying the behavior pattern for the user. The behavior pattern record (394) of FIG. 17 also includes an active field (221) which is a Boolean indicator identifying if the behavior pattern is active, that is, whether the behavior pattern record is presently in use to identify and carry out helpful actions for life support.

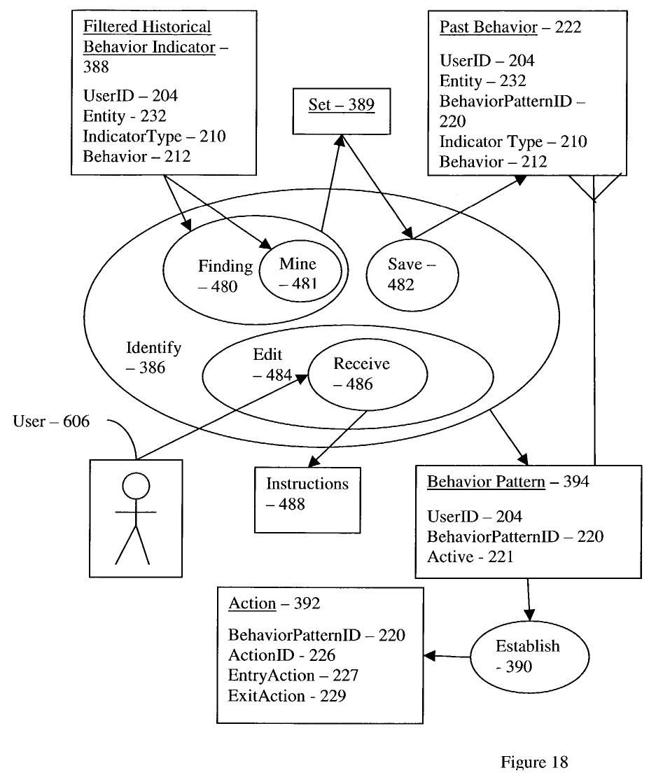

FIG. 18 is a data flow diagram illustrating a method of identifying (386) a behavior pattern for the user. The method of FIG. 18 includes finding (480) a set (389) of filtered historical behavior indicators (388) that identify a behavior pattern (394) for the user and saving (482) the filtered historical behavior indicators (388) as past behavior records (222).

In the method of FIG. 18, saving (482) the set (389) of filtered historical behavior indicators (388) as past behavior records (222) includes saving the filtered historical behavior indicators (388) as past behavior records (222) having a BehaviorPatternID field (220), advantageously adding that field to the records for use in inferring a behavior pattern from past behaviors. Saving (482) the filtered historical behavior indicators (388) as past behavior records (222) also includes saving the past behavior records (222) as records related to the behavior pattern record (394) many to one through the BehaviorPatternID filed (220) used as a foreign key.

Finding (480) a set (389) of filtered historical behavior indicators (388) that identify a behavior pattern (394) for a user includes discovering behavior patterns within the collection of historical behavior indicators. One way of finding (480) a set (389) of historical behavior indicators (388) that identify a behavior pattern (394) for a user within the filtered historical behavior indicators includes data mining (481).

There are many definitions for data mining. For the purposes of this specification, data mining (481) in the method of FIG. 18 means analyzing the filtered historical behavior indicators and discovering relationships, patterns, knowledge, or information from the filtered historical behavior indicators and using the discovered relationships, patterns or knowledge to identify behavior patterns for a user. Many typical data mining techniques include the steps of preparing the data for data mining, choosing an appropriate data mining algorithm, and deploying the data mining algorithm.

In the method of FIG. 18, the filtered historical behavior indicators represent behavior indicators that have been prepared for data mining. That is, the filtered behavior indicators are converted into a predetermined internal data structure when the historical behavior indicators are filtered for a user. The particular predetermined internal data structure will vary depending on factors such as the type of filtered historical behavior indicator, the data mining algorithms used, or any other factor that will occur to those of skill in the art.

Data mining (481) includes choosing an appropriate data mining algorithm for the filtered historical behavior indicators., An appropriate data mining algorithm will vary on many factors such as the type of filtered behavior indicators, the available computer software and hardware used to carry out the data mining, the size of the collection of filtered behavior indicators, or any other factor that will occur to those of skill in the art. Many data mining algorithms exist and all algorithms that appropriately find behavior patterns from a collection of filtered historical behavior indicators are within the scope of the present invention as will occur to those of skill in the art. Although many data mining algorithms exist, many of the data mining algorithms share the same goals. Typical data mining algorithms attempt to solve the problem of being overwhelmed by the volume of data that computers can collect. Data mining algorithms attempt to shield users from the unwieldy body of data by analyzing it, summarizing it, or drawing conclusions from the data that the user can understand.

One way of discussing various data mining algorithms is by discussing the functions that they perform. In this specification, various data mining algorithms are explained by describing the functions that the data mining algorithms perform rather than the specifics of their underlying mathematical operation. Data mining algorithms may also be explained by describing the rule returned by the data mining algorithm. A rule is a description of the relationship, pattern, knowledge, or information found by the data mining algorithm. Data mining algorithms are explained by describing the functions they perform and the rules they return to demonstrate the breadth of the present invention. The classification and description of the following specific examples of data mining algorithms are included in this specification for clarity of discussion, not for limitation. Any method of data mining that will occur to those of skill in the art, regardless of classification, or underlying mathematical operation, that finds behavior patterns for a user from a collection of filtered historical behavior indicators is within the scope of the present invention.

One way of identifying a behavior pattern for a user includes data mining with an association function. Association functions typically are used to find patterns having connected or related events. Data mining with association functions may return a rule describing an affinity or relationship among a collection of filtered historical behavior indicators. An example of a rule returned by an association function is “72% of the filtered behavior indicators that included items A, B and C also contain items D and E.” The specific percentage of occurrences (in this case 72) is called the confidence factor of the rule. Also, in this rule, A, B, and C are said to be on an opposite side of the rule to D and E. Associations can involve any number of items on either side of the rule.

Data mining with an association function may be used to determine an association between the behavior field of the filtered historical behavior indicators and the entity field representing the entity whose behavior resulted in the generation of the filtered historical behavior indicator. For example, an association function may return a rule describing that some percentage of credit card purchases over a certain price are made by a particular entity.

Another method of identifying a behavior pattern for a user includes data mining with sequential pattern operators. Data mining with sequential patterns is typically used in analyzing filtered historical behavior indicators of a single type or generated as a result of the behavior of a single entity. A sequential pattern operator may be used to return a rule that describes a sequential relationship among actions. An example of a rule describing relationship among actions is a rule that identifies purchases that frequently precede other purchases. Data mining with a sequential pattern operator may return a rule demonstrating that the user typically purchased one type of product before or after purchasing another product, or a user traveled to one location before or after traveling to another.

Another method of identifying a behavior pattern for a user includes data mining with a classification operator. A classification operator is applied to a set of filtered behavior indicators that are organized or ‘tagged’ as belonging to a certain class, such as for example, filtered behavior indicators including locations of a user's car. A classification operator examines the set of tagged filtered behavior indicators and produces descriptions of characteristics of the class of tagged filtered behavior indicators. A class description generated by data mining with classification operator may be explicit such as a set of rules describing each class.

A class description generated by data mining with classification operators may also be implicit. An example of an implicit class description is a mathematical function that identifies the class. A mathematical function that identifies the class is a mathematical function which returns the class to which a filtered behavior indicator belongs when a filtered behavior indicator is given as input to the classification operator. The class descriptions generated by data mining with classification operators can be used to tag new filtered behavior indicators to determining which class they belong. The class descriptions are often called a ‘model.’

A classification operator may be used, for examples, to analyze a class of filtered behavior indicator records including locations to determine preferred traveling times for a user. Using filtered behavior indicators including locations, and times of day, a classification operator may determine that times of day for traveling in a car are GOOD, MEDIUM or POOR. An explicit model determines a description of a GOOD, MEDIUM or POOR car traveling situation. Such as a GOOD traveling situation is characterized by X time of day, on Y road, with Z time of travel. An implicit model determines if A time of day, on B road, with C time of travel belongs to a GOOD, MEDIUM, or POOR traveling situation.

Another way of identifying a behavior pattern for a user includes data mining with a clustering operator. By contrast to data mining with a classification operator whose input are a set of tagged filtered behavior indicators, the inputs to a clustering operator are a set of untagged filtered historical behavior indicators. No classes are known at the time the clustering operator is applied. Data mining with a cluster operator may be used to segment or classify the filtered behavior indicator records. Many of the underlying mathematical operations used to build classification operators can also be used to build clustering operators.

In exemplary methods of providing life support services to a user, various data mining algorithms may be used together. For example, an association operator may be used to identify behavior patterns identifying a first set of products that a user often purchases together, and a sequential pattern operator may be used to identify a second set of products that the user often purchases before or after the first set of products.

In some example embodiments, the step of finding (480) a behavior pattern for the user may be carried out by an LSS application (128) running on an LSS server (104) such as the LSS application (128) and LSS server (104) illustrated in FIG. 1. The LSS application includes data mining software similar to existing products such as IBM's “Intelligent Miner.” IBM's “Intelligent Miner” can be operated in several computing environments including AIX, AS/400, and OS/390. The IBM Intelligent Miner is an enterprise data mining tool, designed for client/server configurations and optimized to mine very large data sets, such as gigabyte data sets. The IBM Intelligent Miner includes a plurality of data mining techniques or tools used to analyze large databases and provides visualization tools used to view and interpret the different mining results.

While the method of FIG. 18 has been described in detail with regard to data mining, any method identifying a behavior pattern for a user is within the scope of the present invention, not just data mining. In various exemplary embodiments of the method of the present invention, identifying a behavior pattern for a user includes using data discrimination to identify a behavior pattern for a user, using artificial intelligence to identify a behavior pattern for a user, using machine learning to identify a behavior pattern for a user, using pattern recognition to identify a behavior pattern for a user, or any method of identifying a behavior pattern for a user that will occur to those of ordinary skill in the art.

Identifying a behavior pattern for a user according to the method of FIG. 18 includes editing (484) the past behavior records (222). As described in detail above, many data mining processes are statistical or probabilistic rather than prescriptive and as such can possible find sets of filtered historical behavior indicators for a behavior pattern that in a prescriptive sense have little or nothing to do with the actual behavior represented by a pertinent behavior pattern record. That is, methods used to find a set of filtered historical behavior indicators identifying a behavior pattern for a user may save in the past behavior records filtered behavior indicators which are unnecessary, undesirable, or inefficient in identifying a behavior pattern for a user. Editing (484) the past behaviors (572) advantageously includes manipulating, changing, deleting, adding, or otherwise amending the past behaviors to more accurately or efficiently identify the new behavior pattern.

In the method of FIG. 18, editing (484) the past behavior records (222) includes receiving (486) editing instructions (488) from the user (606). In one example embodiment according to the present invention, receiving (486) editing instructions (488) from the user (606) is carried out by an LSS application (104) running on an LSS server (128) such as those illustrated in FIG. 1. Using a web browser installed on a computer (112) a user may access the past behavior records (222) saved on the LSS Server (104) across an internet (118). A web browser installed on the computer (112) provides an interface for an LSS application (128) running on the LSS server (104) to receive editing instructions from a user. The user may input instructions to edit, add or delete the past behaviors to more accurately identify the behavior pattern. In some example embodiments, the LSS application software prompts the user to input editing instructions, receives the editing instructions, and carries out editing the past behaviors.

Another way the LSS application may carry out receiving (486) editing instructions from a user (606) is by receiving DTMF signals across a circuit switched network such as the telephone network of FIG. 1. A user may input editing instructions telephone key pad. The LSS application (128) receives the editing instructions as DTMF signals and the LSS application carries out editing the past behavior in accordance with the user's instructions.

The method of FIG. 18 also includes establishing (390) an action (392) associated with the behavior pattern (394) for the user. The action (392) association with the behavior pattern (394) for a user according to the method of FIG. 18 operates in the same manner as an action taken in dependence upon a behavior pattern for a user according to the method of FIG. 3. When new behavior pattern (394) is subsequently identified because the filtered behavior indicators for a user match the past behavior records (222) related to the behavior pattern, the action associated with the behavior pattern is identified and executed.

Actions associated with behavior pattern (394) are represented in data structures as records such as the action records (392) of FIG. 18. The action records (392) include a UserID field (204) identifying the user. The action records of FIG. 18 also include a BehaviorPatternID field (220) identifying the behavior pattern (394) for the user associated with the action record (392). Because more than one action may be associated with the new behavior pattern, the action records (392) are related many to one through the UserID field (204) and the BehaviorPatternID field (220) used as a composite foreign key.

The action records of FIG. 18 also include an actionID field (226). The actionID field (226) identifies the action associated with a behavior pattern (394) for the user. In one example, the actionID field (226) may include a filename identifying a file that when run executes the action associated with the new behavior pattern. The action records (392) also include an EntryAction field (227) and an ExitAction field (229) identifying the action as an entry action, an exit action, or both an entry action and exit action.

In some typical embodiments, establishing (390) an action associated with the behavior pattern (394) includes receiving a users' selection from a list of possible actions, displayed with a prompt for a user's selection through a menu on a graphical user interface on a personal computer. The selected action is then associated with the new behavior pattern as an action to be taken in dependence upon the new behavior pattern. As a result, when the new behavior pattern is subsequently identified, the action associated with the new behavior pattern is identified and executed.

Establishing (390) an action (392) associated with the behavior pattern (394) may be carried out by an LSS application (128) running on an LSS server (104) such as, for example, the LSS application (128) running on an LSS server (104) as depicted in FIG. 1. The LSS application (128) may provide a selection of available actions. A user may access the selection of available action across an internet (118) using a web browser installed on a computer (122), or across a circuit switched network such as the telephone network, or any other method of accessing the behavior pattern that will occur to those of skill in the art. In one example, a user may select an action to be associated with the behavior pattern and designate the action as an entry action, an exit action, or both an entry and exit action using a webbrower installed on a computer. The LSS application receives the users selected action and the LSS application associates the selected action with the behavior pattern by creating an action record having an actionID field identifying the selected action, having a behavior patternID identifying the behavior pattern associated with the action, and indicating the action as an entry action or exit action.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

What is claimed is:

1. A method of providing life support services to a user, the method comprising:
receiving a plurality of disparate historical behavior indicators;
filtering the historical behavior indicators for a user in dependence upon user filter attributes, producing filtered historical behavior indicators; and
identifying a behavior pattern for the user in dependence upon the filtered historical behavior indicators.

2. The method of claim 1 wherein identifying a behavior pattern for the user comprises finding a set of filtered historical behavior indicators that identify a behavior pattern for the user.

3. The method of claim 2, further comprising saving the set of filtered historical behavior indicators as past behavior records for the user.

4. The method of claim 2 wherein finding a set of filtered historical behavior indicators that identify a behavior pattern for the user comprises data mining.

5. The method of claim 1, further comprising editing the past behavior records.

6. The method of claim 5, wherein editing the past behavior records comprises receiving editing instructions from the user.

7. The method of claim 1 further comprising establishing an action associated with the behavior pattern for the user.

8. A system for providing life support services to a user, the system comprising:
means for receiving a plurality of disparate historical behavior indicators;
means for filtering the historical behavior indicators for a user in dependence upon user filter attributes, means for producing filtered historical behavior indicators; and
means for identifying a behavior pattern for the user in dependence upon the filtered historical behavior indicators.

9. The system of claim 8 wherein means for identifying a behavior pattern for the user comprises means for finding a set of filtered historical behavior indicators that identify a behavior pattern for the user.

10. The system of claim 9, further comprising means for saving the set of filtered historical behavior indicators as past behavior records for the user.

11. The system of claim 9 wherein means for finding a set of filtered historical behavior indicators that identify a behavior pattern for the user comprises data mining.

12. The system of claim 8, further comprising means for editing the past behavior records.

13. The system of claim 12, wherein means for editing the past behavior records comprises means for receiving editing instructions from the user.

14. The system of claim 8 further comprising means for establishing an action associated with the behavior pattern for the user.

15. A computer program product for providing life support services to a user, the computer program product comprising:
a recording medium;
means, recorded on the recording medium, for receiving a plurality of disparate historical behavior indicators;
means, recorded on the recording medium, for filtering the historical behavior indicators for a user in dependence upon user filter attributes, means, recorded on the recording medium, for producing filtered historical behavior indicators; and
means, recorded on the recording medium, for identifying a behavior pattern for the user in dependence upon the filtered historical behavior indicators.

16. The computer program product of claim 15 wherein means, recorded on the recording medium, for identifying a behavior pattern for the user comprises means, recorded on the recording medium, for finding a set of filtered historical behavior indicators that identify a behavior pattern for the user.

17. The computer program product of claim 16, further comprising means, recorded on the recording medium, for saving the set of filtered historical behavior indicators as past behavior records for the user.

18. The computer program product of claim 16 wherein means, recorded on the recording medium, for finding a set of filtered historical behavior indicators that identify a behavior pattern for the user comprises data mining.

19. The computer program product of claim 15, further comprising means, recorded on the recording medium, for editing the past behavior records.

20. The computer program product of claim 19, wherein means, recorded on the recording medium, for editing the past behavior records comprises means, recorded on the recording medium, for receiving editing instructions from the user.

21. The computer program product of claim 15 further comprising means, recorded on the recording medium, for establishing an action associated with the behavior pattern for the user.
